(19) United States
(12) Patent Application Publication
(10) Pub. No.: US 2025/0090334 A1
(43) Pub. Date: Mar. 20, 2025

(54) LOAD DISTRIBUTION FOR SURGICAL IMPLANTS

(71) Applicant: Board of Supervisors of Louisiana State University and Agricultural and Mechanical College, Baton Rouge, LA (US)

(72) Inventors: Gulshan Neville Sunavala-Dossabhoy, Shreveport, LA (US); Kevin John McCarthy, Shreveport, LA (US)

(21) Appl. No.: 18/809,242

(22) Filed: Aug. 19, 2024

Related U.S. Application Data
(60) Provisional application No. 63/533,269, filed on Aug. 17, 2023.

(51) Int. Cl. A61F 2/36

(52) U.S. Cl. CPC A61F 2/3662; A61F 2002/3625; A61F 2310/00023

(57) ABSTRACT

A femoral stem device is described herein comprising a stem and a neck, wherein the stem comprises an outer shell, wherein the stem comprises a proximal region and a distal region, wherein an interior of the proximal region is selectively hollowed, wherein a loading of the device comprises deforming laterally disposed walls of the proximal region outer shell.

### RELATED APPLICATIONS

This application claims the benefit to U.S. Application No. 63/533,269, filed Aug. 17, 2023.

### TECHNICAL FIELD

The disclosure herein involves femoral stem implant technology, under an embodiment.

### BACKGROUND

In current-generation designs of total primary hip joint replacement, the prostheses are fabricated from alloys. The modulus of elasticity of the alloy is substantially higher than that of the surrounding bone. This discrepancy plays a role in a phenomenon known as stress shielding, in which the bone bears a reduced proportion of the applied load. Stress shielding has been implicated in aseptic loosening of the implant which, in turn, results in reduction in the in vivo life of the implant. Rigid implants shield surrounding bone from mechanical loading, and the reduction in skeletal stress necessary to maintain bone mass and density results in accelerated bone loss, the forerunner to implant loosening. Femoral stems of various geometries and surface modifications, materials and material distributions, and porous structures have been investigated to achieve mechanical properties of stems closer to those of bone to mitigate stress shielding. For improved load transfer from implant to femur, a strategic debulking effort to imparts controlled flexibility while retaining sufficient strength and endurance properties.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### SUMMARY OF THE INVENTION

A femoral stem device is described herein comprising a stem and a neck, wherein the stem comprises an outer shell, wherein the stem comprises a proximal region and a distal region, wherein an interior of the proximal region is selectively hollowed, wherein a loading of the device comprises deforming laterally disposed walls of the proximal region outer shell. In embodiments, the deforming comprises lateral deformation of the walls adjacent to the selectively hollowed spaces.

In embodiments, the selective hollowing comprises an entire hollowing of the proximal region interior.

In embodiments, the selective hollowing comprises a partial hollowing of the proximal region interior.

In embodiments, the partial hollowing provides a curved central spine.

In embodiments, the curved central spine is aligned with a curved longitudinal axis of the stem proximal region.

In embodiments, the partial hollowing comprises beams laterally extending from the central spine.

In embodiments, the central spine and beams define a series of intervening hollowed spaces.

In embodiments, the intervening hollowed spaces comprise laterally opposed internal surfaces.

In embodiments, the intervening hollowed spaces comprise longitudinally opposed internal surfaces.

In embodiments, the deformation transfers at least a portion of the loading into the femur.

In embodiments, the walls comprise a width of 2 mm.

In embodiments, the stem is configured for implantation into the femur.

In embodiments, the distal region of the stem is solid.

In embodiments, the solid stem is partially hollowed to provide a channel for removal of unfused alloy powder.

In embodiments, the neck is configured for attachment to a femoral head.

In embodiments, the femoral stem device comprises Titanium grade 5 (Ti-6Al-4V).

### BRIEF DESCRIPTION OF THE DRAWINGS

The patent or application file contains at least one drawing executed in color. Copies of this patent or patent application publication with color drawing(s) are provided to the Office, including payment of the necessary fee.

FIG. 1. FEA model mesh and materials of implanted femur. (A) Surface view of 3D model. Two kinematic couplings were used: RP1, between the condyles of the distal femur and RP2, at the center of the femoral head. RP1 was fixed in space, while displacement of RP2 was constrained to the vertical axis of the local coordinate system (red arrow). (B) Vertical section of implanted femur. Tie constraints to bond parts together were used between (1) the caudal and cranial halves of the femur, (2) the acetabular cup and the femoral head and neck, and (3) the reamed femur, cement, and implant and the reamed femur and implant. Surface-to-surface contact was between the calcar collar and mating femur surface. (C) FE mesh model of the entire femur and the direction of displacement from the point of applied load (red arrow).

FIG. 2. Coronal sectional views of implants. External dimensions were adapted from the Zimmer M/L Taper Hip Prosthesis with or without the addition of a collar. Various truss implant designs were created and sectional views of collared implants are shown. (A) Proximal hollowed stem. (B-D) Truss designs, FB-2A, FB-3B, and FB-3C. The distal portion of all stems was solid.

FIGS. 3A-3B. Stress distribution in natural femur. A small sphere was embedded at the center of the femoral head and the load and directional constraints were applied at the center of the sphere. As defined in ISO 7206:4:2010 [32], a load of 2300 N was applied to the femoral head. (A) von Mises stress intensity distribution. Regions of significant compressive stress occur on the medial aspect of the femur, above and below the lesser trochanter (arrows). (B) Maximum principal (tensile) stress plot. The largest region of tensile stress occurs along the lateral aspect of the diaphysis, while there is also a noticeable region of tensile stress at the femoral neck besides the greater trochanter (block arrows).

FIGS. 4A-4E. von Mises stress intensity in a femur with collared stems implanted in bone contact. (A) Solid, (B) hollow, (C) FB-2A, (D) FB-3B, and (E) FB-3C. Proximal medial region of stress shielding with solid implant (red frame). Increase in stress returned to the proximal medial bone with FB-3B and FB-3C implants (arrows). The stress plots are set between 0 and 20 MPa for comparative purposes.

FIGS. 5A-5E. Maximum principal (tensile) stresses in femur with collared stems implanted in interference fit. (A) Solid, (B) hollow, (C) FB-2A, (D) FB-3B, and (E) FB-3C.

FIGS. 6A-6D. Stress in femur with collarless stems in interference fit. (A,B) von Mises stress and (C,D) maximum principal stress in femur. (A,C) Solid implant and (B,D) FB-3C implant. Region of stress shielding with solid implant (red frame). Noticeably less stress shielding at proximal medial interface irrespective of a collarless FB-3C stem (arrows).

FIGS. 7A-7E. Maximum principal stresses in collared Ti-6AL-4V implants when in contact with bone. (A) Solid, (B) hollow, (C) FB-2A, (D) FB-3B, and (E) FB-3C. Arrows show regions of peak stress. All stress plots are set between 0 and 100 MPa for comparative purposes.

FIG. 8. von Misses stress intensity in proximal femur. von Misses stress values plotted in cranial-caudal direction at the (A) medial, (B) lateral, (C) anterior, and (D) posterior interference face. Line traces of solid, FB-3B, and FB-3C implants are overlayed in the graphs.

FIG. 9. Stress intensity in a proximal femur implanted with collared and collarless stems of solid and truss configurations. (A) The Gruen reference zones. Nodal stress values were averaged across 5 mm segments in cranial-caudal direction of the reamed bone cavity. (B) Minimum principal stress (compressive) in medial bone (Gruen zone 7), and (C) maximum principal stress (tensile) in proximal lateral bone (Gruen zone 1).

FIG. 10. von Mises stress ratio in proximal femur. The percentage change in stress was calculated by using stress intensity averaged across equally spaced proximal distance at implant interfaces of solid, collared FB-3B, and collared FB-3C implants and, correspondingly, in the intact femur. Stress ratio in (A) proximal medial femur (Gruen zone 7) and (B) proximal lateral femur (Gruen zone 1).

FIGS. 11A-11F. Endurance testing with ISO standard set-up. (A,B) Static load tests of solid and FB-3C implants. Gross yielding of neck region in solid implant and buckling of outer casing below trusses in the FB-3C implant (block arrows). Equivalent plastic strain (PEEQ) is set between 0 and 0.0140. (C,D) Maximum principal stresses in endurance tests of solid and FB-3C implants. Maximum principal stress is set between 0 and 50 MPa. (E) Expanded sectional view of FB-3C implant. Region of peak tensile stress at condylar head-neck junction in solid implant and at lower trusses in FB-3C implant (arrows). (F) Additively manufactured Ti-A16-4V truss implant, FB-3B, using laser powder bed fusion-external and vertically sectioned views are shown.

FIGS. 12A-12F. Embodiments of a femoral stem device.

FIGS. 13A-13E. von Mises stress intensity in femur with cemented collared stems. (A) Solid, (B) hollow, (C) FB-2A, (D) FB-3B, and (E) FB-3C. Stress plots are set between 0-20 MPa for comparative purposes. Stress shielding in medial proximal bone (red frame). Increase in stress at proximal medial femur with debulked implants (arrows).

FIGS. 14A-14E. Maximum principal (tensile) stresses in femur with cemented collared stems. (A) Solid, (B) hollow, (C) FB-2A, (D) FB-3B, and (E) FB-3C. Stress plots are set between 0-20 MPa for comparative purposes. A localized increase in tensile stress at the proximal lateral connection between cement and femur (arrow).

FIGS. 15A-15E. Maximum principal (tensile) stress at bone-cement interface with collared stems. A. Solid, B. hollow, C. FB-2A, D. FB-3B, E. FB-3C. Stress plots are set between 0-2.4 MPa for comparative purposes. Peak stress (arrows) with debulked implants occur at proximal lateral bone-cement interface.

FIGS. 16A-16E. Maximum principal stress in collared stems when cemented. (A) Solid, (B) hollow, (C) FB-2A, (D) FB-3B, and (E) FB-3C. Stress plots are set between 0-100 MP a for comparative purposes.

FIGS. 17A-17D. Stress in femur with cemented collarless stems. (A, B) von Mises stress and (C, D) maximum principal stress. (A, C) Solid implant. (B, D) FB-3C implant. Stress plots are set between 0-20 MPa for comparative purposes. Noticeably higher compressive stress at proximal medial bone with FB-3C implant than solid implant (arrow).

FIGS. 18A-18D. Maximum principal stress intensity in collarless stems and at cement-bone interface. (A,B) Tensile stresses at cement interface with solid implant and FB-3C implant. (C,D) Tensile stress in collarless solid and FB-3C implants. Peak stress (arrows) in collarless implants-solid: 180 MPa without collar compared to 182 with collar; FB-3C: 194 MPa without collar compared to 159 MPa with collar.

FIGS. 19A-B. Compressive and tensile stress in proximal femur implanted with collared solid, FB-3B, and FB-3C stems. Nodal stress values were averaged across 5 mm segments in cranial-caudal direction of the reamed bone cavity. (A) Minimum principal stress (compressive) in medial bone (Gruen zone 7), and (B) maximum principal stress (tensile) in proximal lateral bone (Gruen zone 1).

FIG. 20. The Vancouver classification of periprosthetic fractures of the femur about a total hip arthroplasty (Image from [5])

FIG. 21. Two Javid E-p Results Compared against Seven E-p Equations (FIG. 4.10 of [8])

FIG. 22. S-N curves in Tl-6Al-4V. Effect of degree of texture.

FIG. 23. FEA Model Mesh and Materials (Left. Full 3D Model; Right. Cross-Section View)

FIG. 24. Measurements for Alpha and Beta per ISO 7206-4:2010 (Figure A.3)

FIG. 25. Hollow and Fish bone Cross-Sectional Views

FIG. 26A. Natural Femur-von Mises Stress Intensity Plot

FIG. 26B. Natural Femur-Maximum (Tensile) Principal Stress Plot

FIGS. 27A-1-27A-7. Von Mises Stress Intensity in Implanted Femur with Bone/Ti Contact

FIGS. 27B-1-27B-7. Maximum Principal (Tensile) Stresses in Implanted Femur with Bone/Ti Contact

FIGS. 28A-1-28A-3. Von Mises Stress Intensity in Implanted Femur-Cemented

FIGS. 28B-1-28B-3. Maximum Principal (Tensile) Stresses in Implanted Femur-Cemented

FIGS. 29A-29C. Maximum Principal Stresses at Cement-Bone Interface

FIGS. 30A-30C. Maximum Principal Stresses in Ti-6Al-4V Implant when Cemented

FIGS. 31A-31C. Maximum Principal Stresses in Ti-6Al-4V Implant when in Contact with Bone

FIG. 32A. No Medial Collar-Cemented-von Mises Stress Intensities

FIG. 32B. No Medial Collar-Cemented-Maximum Principal Stresses

FIG. 33A. No Medial Collar-Bone/Ti-von Mises Stress Intensities

FIG. 33B. No Medial Collar-Bone/Ti-Maximum Principal Stresses

FIG. 34. No Medial Collar-Cement-Bone Bond-Maximum Principal Stresses

FIG. 35. No Medial Collar-Implant-Maximum Principal Stresses

FIG. 36. Static Load Tests-Solid Implant

FIG. 37. Static Load Tests—FB-3B Implant

FIG. 38. Static Load Tests—FB-3C Implant

FIG. 39. Endurance Tests—Solid Implant—Maximum Principal Stresses

FIG. 40A. Endurance Tests—FB3C Implant—Maximum Principal Stresses

FIG. 40B. Endurance Tests—FB3C Implant—Maximum Principal Stresses

FIG. 41A shows an external view of a screw-based dental implant.

FIG. 41B shows a vertical sectional view of a screw-based dental implant.

FIG. 41C shows a vertical sectional view of a screw-based dental implant under applied occlusal load.

### DETAILED DESCRIPTION

### Example 1

In current-generation designs of total primary hip joint replacement, the prostheses are fabricated from alloys. The modulus of elasticity of the alloy is substantially higher than that of the surrounding bone. This discrepancy plays a role in a phenomenon known as stress shielding, in which the bone bears a reduced proportion of the applied load. Stress shielding has been implicated in aseptic loosening of the implant which, in turn, results in reduction in the in vivo life of the implant. Rigid implants shield surrounding bone from mechanical loading, and the reduction in skeletal stress necessary to maintain bone mass and density results in accelerated bone loss, the forerunner to implant loosening. Femoral stems of various geometries and surface modifications, materials and material distributions, and porous structures have been investigated to achieve mechanical properties of stems closer to those of bone to mitigate stress shielding. For improved load transfer from implant to femur, a strategic debulking effort to imparts controlled flexibility while retaining sufficient strength and endurance properties. Using an iterative design process, debulked configurations based on an internal skeletal truss framework were evaluated using finite element analysis. The implant models analyzed were solid; hollow, with a proximal hollowed stem; FB-2A, with thin, curved trusses extending from the central spine; and FB-3B and FB-3C, with thick, flat trusses extending from the central spine in a balanced-truss and a hemi-truss configuration, respectively. As outlined in the International Organization for Standardization (ISO) 7206 standards, implants were offset in natural femur for evaluation of load distribution or potted in testing cylinders for fatigue testing. The commonality across all debulked designs was the minimization of proximal stress shielding compared to conventional solid implants. Stem topography can influence performance, and the truss implants with or without the calcar collar were evaluated. Load sharing was equally effective irrespective of the collar; however, the collar was critical to reducing the stresses in the implant. Whether bonded directly to bone or cemented in the femur, the truss stem was effective at limiting stress shielding. However, a localized increase in maximum principal stress at the proximal lateral junction could adversely affect cement integrity. The controlled accommodation of deformation of the implant wall contributes to the load sharing capability of the truss implant, and for a superior biomechanical performance, the collared stem should be implanted in interference fit. Considering the results of all implant designs, the truss implant model FB-3C was the best model.

### 1. INTRODUCTION

Prosthetic hip replacement is commonly performed for end-stage hip pathologies including osteoarthritis of the joint, avascular necrosis of the femoral head, and proximal femoral fractures with underlying osteoporosis. Damaged hip joints are replaced with artificial prostheses made of biocompatible materials. The procedure is increasingly performed due, in part, to age-related or obesity-associated osteoarthritis and to the success of the treatment in providing relief from pain and restoring stability to the joint [1].

Despite evidence that hip arthroplasty is an excellent treatment option for hip replacement, bone loss around the implants is a concern as mechanical disengagement of the implant occurs frequently, which necessitates revision surgeries [2,3]. Most femoral stems are made of solid metals such as titanium, titanium alloys, cobalt-chrome, or stainless steel that have a modulus of elasticity that is markedly higher than that of the contiguous bone [4]. Increased stiffness of the implant results in inadequate load transfer from implant to bone. The decrease in mechanical loading of bone is referred to as stress shielding [5]. Stress shielding is the primary cause of reduced bone mineral density and the sequelae of bone resorption and implant loosening following hip replacement. Aseptic loosening is the loss of mechanical fixation of the femoral stem that is not due to infection. It is one of the more common shortcomings of hip arthroplasties [6], and revision surgeries are more complex due to the poor quality of residual bone which risks intra- and peri-operative fractures. In essence, stress-shielding and associated implant instability (large bone-implant interfacial micromotion) are among the factors that play important roles in aseptic loosening of an implant and, hence, in the decrease in the in vivo life of a joint replacement.

The design of the femoral stems and the choice of material (hence, stiffness of the material) are important considerations in limiting stress-shielding. Various stem geometries and implant materials have been investigated in an effort to increase load on peri-implant bone and suppress the decline in bone stock. The outer characteristics of the stem, such as length [7], taper [8], collar [9], and surface condition [10], have been assessed for improved bone fixation and osseointegration, but their limited advantages to promoting load transfer have redirected attention to stem materials and material distribution to achieve the desired mechanical characteristics [11-16]. Materials of low modulus of elasticity improve load transfer, but undesired flexibility and higher interface stresses could result in the implant debonding from bone [17]. In accordance, the poor clinical performance of isoelastic stems was attributed to detrimental micromotion, increased debris, and premature loss of prosthesis anchorage [18,19]. Stems with a graded modulus of elasticity, achieved through physico-mechanical alterations of material properties such as heat treatment of Ti-Nb6-Sn4 alloys, show improved load transfer capability [20,21]. Nonetheless, achieving the precise spatial distribution of stiffness, through localized heat treatment for graded metal hardening can be challenging. Non-homogenously distributed porosities or rationally graded porosities and lattice structural designs to modulate the stiffness of the stem are being explored [12-16,22]. Topologically graded porous designs reduce stem stiffness while a relatively dense core provides the necessary structural strength for load bearing. Porous structures also afford a path for bone ingrowth to improve implant anchorage. However, pore size and pore interconnectedness are critical determinants for bone formation [23,24], and highly porous implants are conducive to bone invagination but have poor mechanical strength. Achieving the right balance between pore size, degree of porosity, and cross-sectional distribution of stiffness for expected implant performance can be demanding.

Auxetic porous structures exhibit a negative Poisson's ratio, and because of their distinctive architectural unit, they expand perpendicular to axial tension [24,25]. There is a growing interest in adopting them in femoral stem designs as auxetic biomaterials could improve implant-bone contact [24-26]. Loads acting on the implant generate tensile stresses along the lateral interface, and stems with auxetic structures that are distributed where tensile stresses dominate could improve implant-bone contact [27,28]. However, auxetic structures are limited at load transfer in regions of compressive stresses and could exacerbate stress shielding. Stems that accommodate the precise cross-sectional distribution of mechanical stiffness and flexibility facilitate physiological load bearing. The aim of the present study was to explore the effects of interior debulking of the femoral stem on load sharing in the femur.

### 2. MATERIALS AND METHODS

### 2.1. The Femur and Material Property Derivation

The deidentified CT scans of femurs of a 70-year-old patient were converted to 3D CAD models using 3DSlicer. The model was imported into the Autodesk Meshmixer and mesh tessellation was performed to heal, smoothen, and refine irregularities. Internal smoothening was carried out using CAD editing features in the Start CCM+ program. The model was imported in Abaqus 2022 HF3 finite element analysis (FEA) program and was converted to a finite element (FE) model. Using Bonemat software, variable density (p) and variable modulus of elasticity (E) were mapped from the CT scan data of the femur to the FEA model. Three E-p relationships (MPa) were evaluated and Equations (2) and (3) produced similar results.

$$Empirical equation E = 14 , 664 ρ {ash}^{1.49} . \qquad [ 1 ]$$

$$Elasticity equation for human femoral neck E = 6850 ρ {ash}^{1.49} . \qquad [ 2 ]$$

$$Elasticity equation derived in Javid Dissertation E = 11 , 644 ρ {ash}^{1.31} . \qquad [ 3 ]$$

Two representative density values (0.5 and 1.2 g/cm³) were taken from the Bonemat exported data file, and corresponding E values with these densities were plotted against the curves of seven E-p equations [30]. Bone mass/density for humans covers a wide range of values, and by validation, equation (3) was found to be the best representative for the current study. The patient's CT scan did not include the entire length of the femur, and the distal extension was modeled using visual references from a full human femur (NIH 3D Print Exchange) and was assigned generic bone properties (density=0.31 g/cm³, E=14,700 MPa, v=0.3).

### 2.2. Femoral Implant Material Properties

Titanium grade 5 (Ti-6A1-4V) was used for all femoral implant components. Ti-6Al-4V was evaluated for fatigue failure using “fatigue curves”, specific for material, fabrication processes, grain-orientation, testing medium, texture, etc. For Ti-6Al-4V, the worst published texture curve in air was used [31], and based on the fatigue curve, it was determined that at a 10 million cycle run-out, an alternating amplitude stress of 600 MPa is required.

A Keq factor is used for Ti-6Al-4V to account for saline environment testing, manufacturing technique and quality, and other potential surface irregularities. A single “Keq” factor is created from the various contributing “k” factors and is derived from numerous comparisons of the FEA results to laboratory test results for particular products and manufacturing processes, and vendors. A conservative Keq factor of 5 was used in the current study.

### 2.3. FEA Setup

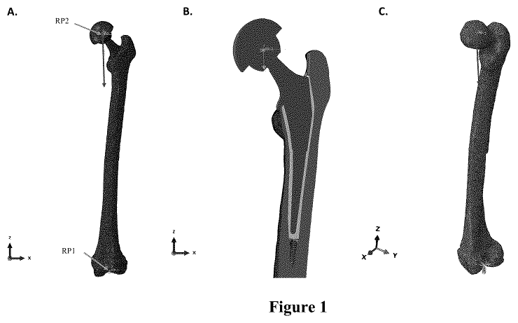

The FEA mesh model of the left femur was cut and reamed to allow for insertion of the femoral implant, with an intervening cement layer assigned E=2300 MPa and Poisson's ratio (v)=0.3. (FIG. 1). The cement region, with its higher quality elements, was changed to bone properties for evaluating direct implant-bone contact. Bonemat divided the density/elasticity properties for cancellous bone at the reamed center of the femur into >300 material layers. For the purposes of uniformity, mean values were used for bone that replaced the intervening cement layer. The medium Bonemat properties (Mat-155) assigned to bone adjoining the implant included: density=0.764 g/cm³, E=8237 MPa, Poisson's ratio (v)=0.3. In the models, the femoral head of the implant and the acetabular cup (without the acetabular cup inlay) were included as a single unit.

FIG. 1 shows EA model mesh and materials of implanted femur. (A) Surface view of 3D model. Two kinematic couplings were used: RP1, between the condyles of the distal femur and RP2, at the center of the femoral head. RP1 was fixed in space, while displacement of RP2 was constrained to the vertical axis of the local coordinate system (red arrow). (B) Vertical section of implanted femur. Tie constraints to bond parts together were used between (1) the caudal and cranial halves of the femur, (2) the acetabular cup and the femoral head and neck, and (3) the reamed femur, cement, and implant and the reamed femur and implant. Surface-to-surface contact was between the calcar collar and mating femur surface. (C) FE mesh model of the entire femur and the direction of displacement from the point of applied load (red arrow).

### 2.3.1. Meshing, Material Models, Part Tie-Constraints, and Part-to-Part Contact

Quadratic tetrahedral meshing was used for all devices, except for the acetabular cup which was swept linear hexahedral meshed. The femur and the femoral head of the implant were meshed with a 1 mm mesh size, while the femoral body and cement were with a 0.75 mm mesh size. All metal components were of Ti-6A1-4V with a linear strain hardening plasticity that had a yield strength of 880 MPa and the ultimate true stress of 1100 MPa and an ultimate true strain of 0.014. Poisson's ratio (v) for all materials was 0.3. Part tie-constraints were used to bond parts together, while surface-to-surface contact was used between the calcar collar and the mating femur surface.

Due to the triangulated import of the femur, a high global 1 mm mesh density was required for acceptable mesh element quality. Experience with previous mesh sensitivity work has shown that such a high mesh density with the use of quadratic (mid-side nodes) formulation produces stress results at or near mesh-converged solutions, typically within a 1% error. The implant, however, with its numerous small features requires strategic mesh refinement or a small global mesh size. For mesh sensitivity analysis, the curvature at the junction of the head and the neck, a location common to the solid and all iterative implant designs, was analyzed. The mesh element type was quadratic tetrahedral, and the global mesh density was set to 0.5 mm with a 0.02 curvature control. Coarser global mesh sizes (0.6 mm and 0.7 mm), as well as highly localized mesh refinements (0.4 mm, 0.3 mm, and 0.2 mm) were included in the sensitivity analysis. Above the global 0.7 mm and below the 0.2 mm mesh sizes, valid meshes were not able to be created. All mesh levels tested were within 0.7% of the converged solution of 59 MPa. More specifically, the as-run case, 0.5 mm mesh size, was within 0.2% of the converged solution.

### 2.3.2. Boundary Conditions, Couplings, and Spatial Model Constraints

Two kinematic couplings were used in the FEA model. The first coupling (RP1) had the lateral and medial condyles of the distal femur, which were modeled with embedded spheres, controlled by a reference point centered between them. The second coupling (RP2) had the outer surface of the acetabular cup controlled by a reference point located at the center of the femoral head. Loading and displacement-controlled constraints, as assigned to a local Cartesian coordinate section, were applied to the RP2. RP2 movement was limited to the vertical axis of the local coordinate system. RP1 was fixed in space with displacement and rotation of both embedded spheres being restricted (FIG. 1).

### 2.3.3. Loads and Creation of Load Direction in the Local Coordinate System

The loading of the natural femur was accomplished by embedding a small sphere, centered in the femoral head. The load and directional constraint were applied to a reference point at the center of the sphere, controlling its motion. Model loads were applied in accordance with the ISO 7206 Standards, with ISO 7206-4:2010 being the most applicable for fatigue conditions and ISO 7206-10:2018 for static load rating [32-34]. A load of 2300 N was applied to the femoral head, following the vertical direction of the local coordinate system as defined in ISO 7206-4:2010 [32].

The orientation of the implanted femoral stem was as specified in ISO 7206-4:2010 [32]. The α angle, the angle in the frontal plane between the load axis and the stem axis, and the β angle, the angle in the lateral plane perpendicular to the frontal plane between the load axis, and the stem axis, were 10° and 9°, respectively. In brief, various axes and planes were used to make the α angle determination in the 3D CAD model using a trigonometric relationship. Due to the irregular spatial positioning of the femurs in the original CT data, the β angle was measured directly in the 3D CAD program as the angle in the lateral plane perpendicular to frontal plane between the load axis and the stem. A new coordinate was established by rotating both a and B by the prescribed amounts to achieve ISO 7206-4:2010 requirements. A load of 2300 N was applied along the local vertical axis for single-direction loading. The new coordinates were to simply provide the necessary line of action for application of load in the local coordinate system (FIG. 1).

In point-to-failure experiments, the distal aspect of the implant stem was potted in a test cylinder using Delrin (density=1.41 g/cm³, E=3100 MPa, and v=0.35) as the potting material [34]. Loading was performed as described in ISO 7206-4:2010 [32], but in a displacement-controlled manner until failure [34]. The acetabular cup was forced to follow the vertical direction in the local coordinate system for an arbitrary 50 mm, and a load-vs-displacement chart was created and the failure load was determined. Implant endurance load testing was performed similarly in a potted test cylinder, but instead of a static load, a cyclic load of 2300 N was applied [15,35] and the implant movement was restricted to the vertical direction of the local coordinate system.

### 2.3.4. Solid, Hollow, and Truss Implant Model Descriptions

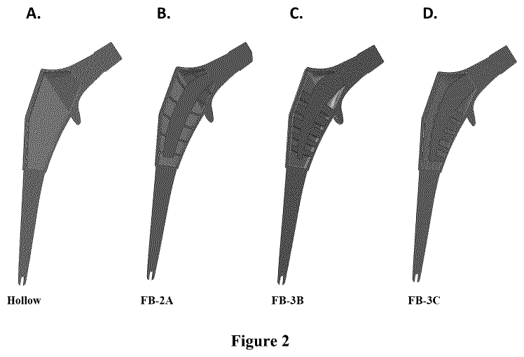

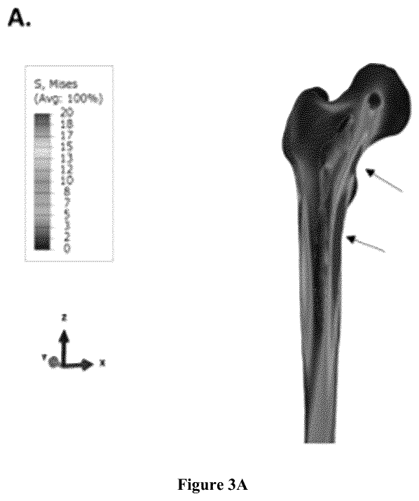

External dimensions of the Zimmer M/L Taper Hip Prosthesis were used for the femoral stem and adapted, or not, with a calcar collar. The solid implant version was followed by the creation of a hollowed-out implant with wall thickness of 2 mm. An internal structure was created with trusses that braced the central spine. The central spine was disconnected from the distal aspect of the stem. Modifications to the spine and trusses included changes in shape and thickness. The distal part of the femoral stem was solid across all implant configurations (FIG. 2).

FIG. 2 shows coronal sectional views of implants. External dimensions were adapted from the Zimmer M/L Taper Hip Prosthesis with or without the addition of a collar. Various truss implant designs were created and sectional views of collared implants are shown. (A) Proximal hollowed stem. (B-D) Truss designs, FB-2A, FB-3B, and FB-3C. The distal portion of all stems was solid.

### 3. RESULTS

### 3.1. Stresses in Intact Femur

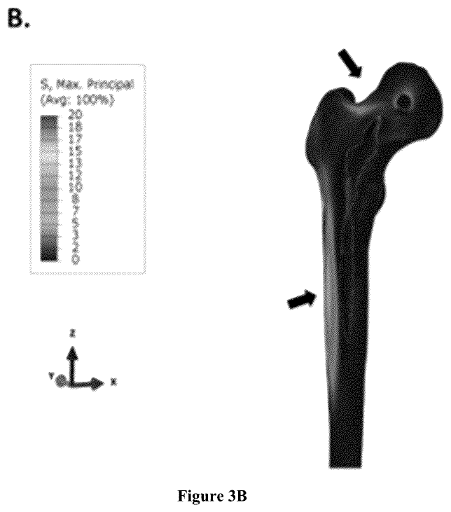

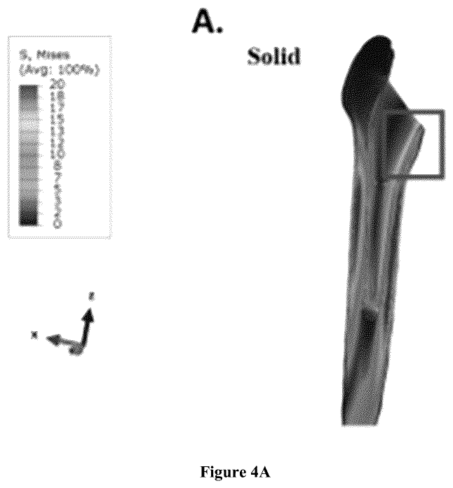

The general distribution of stress after unidirectional loading of the natural femur resulted in higher compressive stress at the medial side of the femur, beginning above the lesser trochanter and extending distally to the medial diaphysis (FIG. 3A). In contrast, a noticeable increase in tensile stress was observed at the lateral side of diaphysis and at the superior aspect of the neck where it unites with the greater trochanter (FIG. 3B).

FIG. 3 shows stress distribution in natural femur. A small sphere was embedded at the center of the femoral head and the load and directional constraints were applied at the center of the sphere. As defined in ISO 7206:4:2010 [32], a load of 2300 N was applied to the femoral head. (A) von Mises stress intensity distribution. Regions of significant compressive stress occur on the medial aspect of the femur, above and below the lesser trochanter (arrows). (B) Maximum principal (tensile) stress plot. The largest region of tensile stress occurs along the lateral aspect of the diaphysis, while there is also a noticeable region of tensile stress at the femoral neck besides the greater trochanter (block arrows).

### 3.2. Stresses in Implanted Femur

FEA simulations were conducted for the solid, hollow, and different truss implants iterations under identical setup and loading conditions. Two sets of simulations were conducted for each implanted femur: first, with direct implant-bone bonding and second, with a cement layer between the reamed bony canal and the implant.

3.2.1. Uncemented vs. Cemented Stems

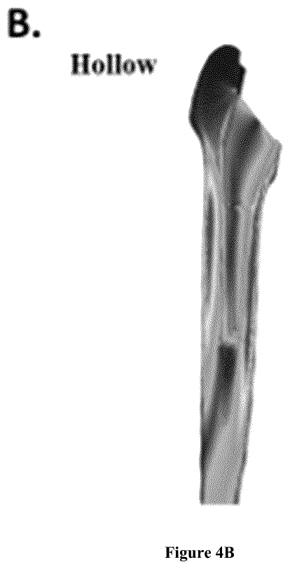

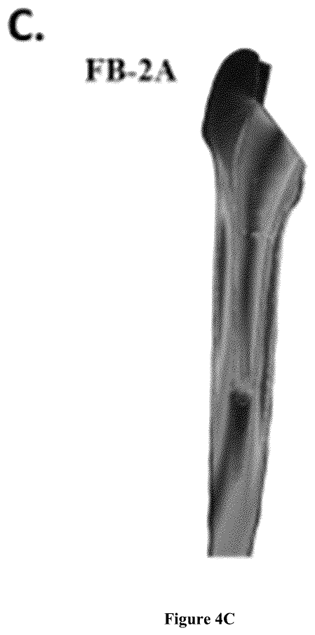

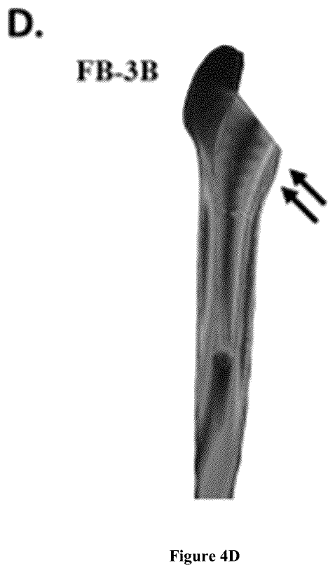

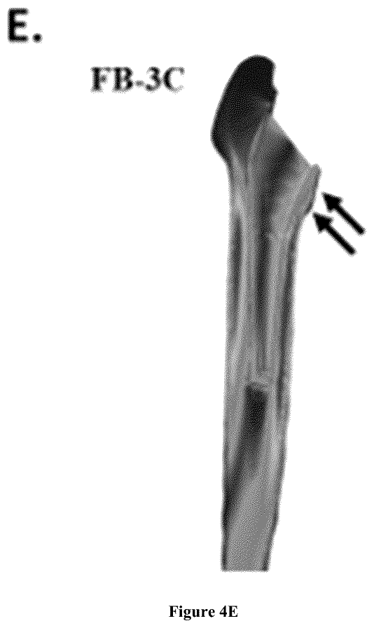

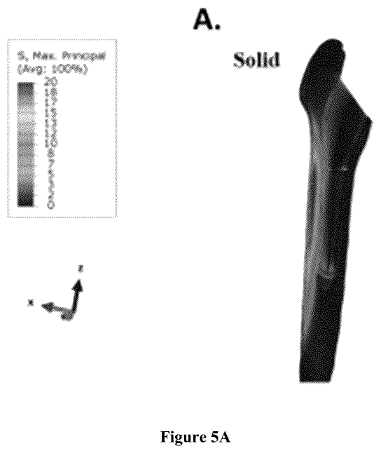

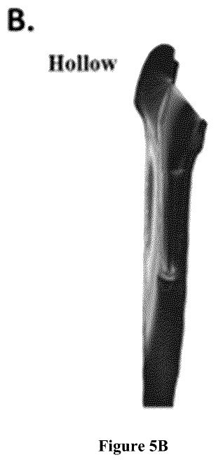

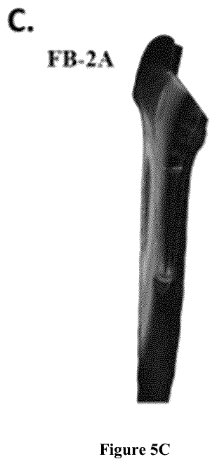

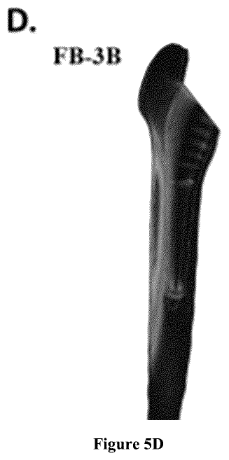

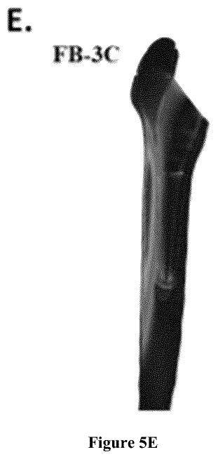

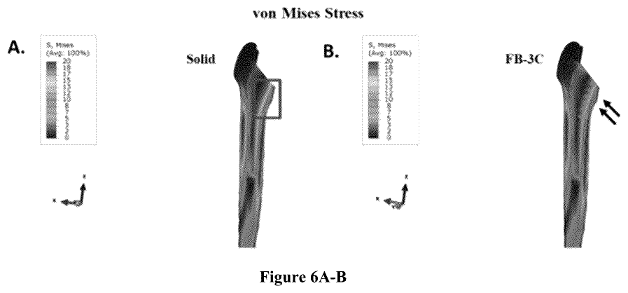

In a femur that was contiguous with the collared implants, there was negligible stress in proximal bone with the solid design (FIG. 4A). The benefit of implant debulking in mitigating stress shielding was clear. Debulking, in general, returned stresses to the region (FIG. 4B-E). In implants with thicker trusses, FB-3B and FB-3C, stress distribution extended to the anterior and posterior sides of the femur compared to the thinner truss implant, FB-2A (FIG. 4). An increase in tensile stress localized to the proximal lateral junction was observed with the hollow and truss implants compared to the solid implant (FIG. 5). The peak tensile stress was the same for all truss implants (26 MPa; FIG. 5C-E), while the hollow design showed a higher peak stress (FIG. 5B).

FIG. 4 shows von Mises stress intensity in a femur with collared stems implanted in bone contact. (A) Solid, (B) hollow, (C) FB-2A, (D) FB-3B, and (E) FB-3C. Proximal medial region of stress shielding with solid implant (red frame). Increase in stress returned to the proximal medial bone with FB-3B and FB-3C implants (arrows). The stress plots are set between 0 and 20 MPa for comparative purposes.

FIG. 5 shows maximum principal (tensile) stresses in femur with collared stems implanted in interference fit. (A) Solid, (B) hollow, (C) FB-2A, (D) FB-3B, and (E) FB-3C.

Cement has a low modulus of elasticity, and it can effectively redistribute the load to the femur. Not surprisingly, the mitigation of stress-shielding was significant across all stems when they were cemented in the femur (Supplementary Figure S1). There was, however, greater load sharing in the proximal region with hollow and truss stem designs in comparison to the solid. The tensile stress profiles were similar across the different designs with the highest tensile stresses located at the lateral diaphysis (Supplementary Figure S2). Of note, a highly localized area of tensile stress was observed at the proximal lateral connection between the cement and the femur when hollow or truss designs were implanted (Supplementary Figure S2B-E). The hollow design resulted in a higher tensile stress in this region, and differences in flexural rigidity of the truss design resulted in changes in stress at the location; specifically, the greater the implant stiffness, the lower the tensile stress.

The tensile stress at the bone-cement interface was lower than that of the Ti-6Al-4V alloy-cement interface. The mean tensile stress at the bone-cement interface was determined to be 6.3 MPa, with a minimum test value of 2.4 MPa and a maximum of 10.2 MPa [36]. To study stress at the cement interface (the surface bonded to bone in the FEA model), the cement layer was isolated and examined using the maximum principal stress. This stress was plotted, on a scale of 0-2.4 MPa, the lower bound for the bone-cement tensile stress as determined in [36]. The results showed that the stiffer the implant, the lower the maximum tensile strength at the bone-cement interface: 4.1 MPa for the solid, 4.4 MPa for the stiffest truss implant (FB-3B), 4.5 MPa for the remaining truss implants (FB-2A and FB-3C), and 5.3 MPa for the hollow design (Supplementary Figure S3). A significant increase in tensile stress above the minimum debonding strength of 2.4 MPa was observed with all implant types. Unlike the solid implant, higher peak tensile stress for the hollow and truss implants were located at the proximal lateral junction (Supplementary Figure S3B-E). As cement has a low bond strength, it can be readily damaged with implants of lower stiffness. High tensile stress at the proximal lateral junction with debulked designs could de-bond the cement from the femur, resulting in premature implant loosening.

3.2.2. Collared vs. Collarless Femoral Stem

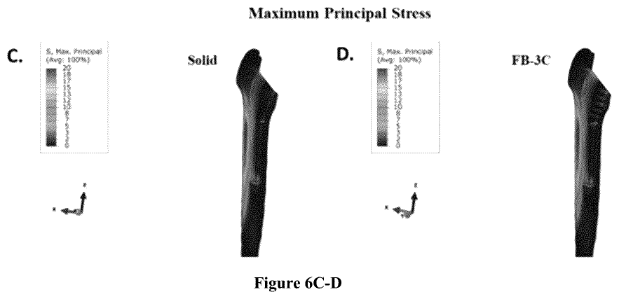

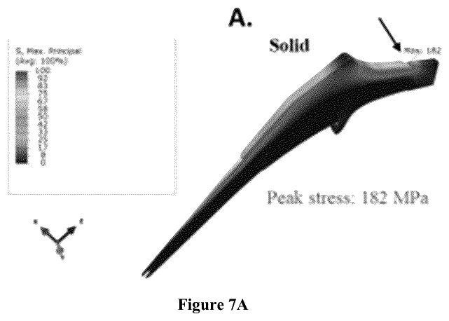

To examine the impact of the calcar collar on stress distribution in bone, the solid and truss implants without the calcar collar were investigated. Despite the absence of a calcar collar, the FB-3C truss implant, when placed in a direct interference fit, improved load sharing in the proximal bone compared to the solid implant (FIG. 6A,B). The maximum principal stress in bone between the collared and collarless solid and truss stems was near-comparable (FIGS. 5 and 6C,D). Alternatively, no differences in von Mises stress intensity and maximum principal stress in the femur were observed between the collarless solid or FB-3C stems when cemented (Supplementary Figure S5). There was, however, a moderate increase in cement-bone bonding stress with the collarless stems compared to stems with calcar collars (solid collarless: 4.3 MPa vs. solid collared: 4.1 MPa; FB-3C collarless: 4.8 MPa vs. FB-3C collared: 4.5 MPa) (FIG. 3 and Supplementary FIG. 6SA,B). Higher stresses in the cement layer with collarless stem geometry could disrupt bonding to bone and, therefore, cementing was considered less desirable.

FIG. 6 shows stress in femur with collarless stems in interference fit. (A,B) von Mises stress and (C,D) maximum principal stress in femur. (A,C) Solid implant and (B,D) FB-3C implant. Region of stress shielding with solid implant (red frame). Noticeably less stress shielding at proximal medial interface irrespective of a collarless FB-3C stem (arrows).

### 3.3. Maximum Principal (Tensile) Stress in Implant

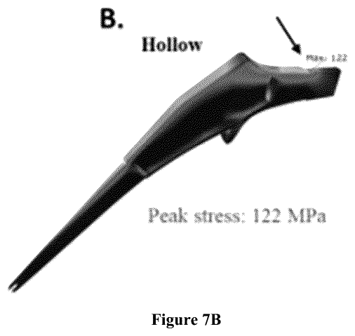

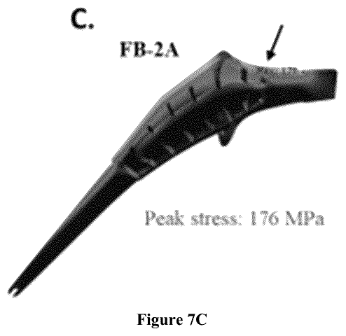

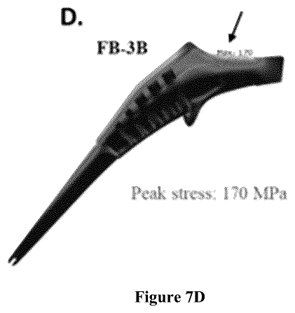

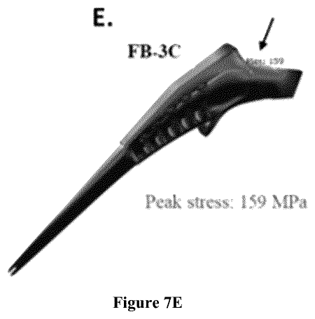

To screen for implant fatigue, maximum principal stresses in the implant were analyzed. Whether or not the collared implants were directly bonded to or cemented in femur, peak stresses at the implant neck were lowest in the most flexible design, the hollow stem (122 MPa; FIG. 7 and Supplementary Figure S4). On the other hand, the stiffest design, the solid stem, showed the highest peak stress in uncemented and cemented conditions at 182 MPa and 184 MPa, respectively. The truss designs with variations in flexural stiffness had peak stresses between the extremes of the solid and hollow implants irrespective of the cementation. Of note, in the interference fit, the area of peak stress was at the junction of the condylar head and neck in solid and hollow designs, while it was at the proximal lateral commissure of the central spine in truss designs (FIG. 7). Among the truss designs, the FB-3C implant showed the lowest peak tensile stress in the cemented and uncemented conditions: cemented—158 MPa and uncemented—159 MPa (FIG. 7 and Supplementary Figure S4). As lower stress in implant can extend service life, FB-3C was considered the most favorable truss design.

Importantly, the absence of the calcar collar resulted in a significant increase in stresses in the cemented FB-3C implant (Supplementary Figure S6D). Compared to the peak tensile stress of 159 MPa in the collared stem, stress increased to 194 MPa in the collarless state (FIG. 7 and Supplementary Figure S6D). The maximum principal stress in the collarless cemented FB-3C was higher than the solid implant of similar external geometry (194 MPa vs. 180 MPa; Supplementary Figure S6C,D). These results suggest that the calcar collar is critical to the performance and life of the truss implant.

FIG. 7. shows maximum principal stresses in collared Ti-6AL-4V implants when in contact with bone. (A) Solid, (B) hollow, (C) FB-2A, (D) FB-3B, and (E) FB-3C. Arrows show regions of peak stress. All stress plots are set between 0 and 100 MPa for comparative purposes.

### 3.4. Quantification of Stress in Proximal Bone

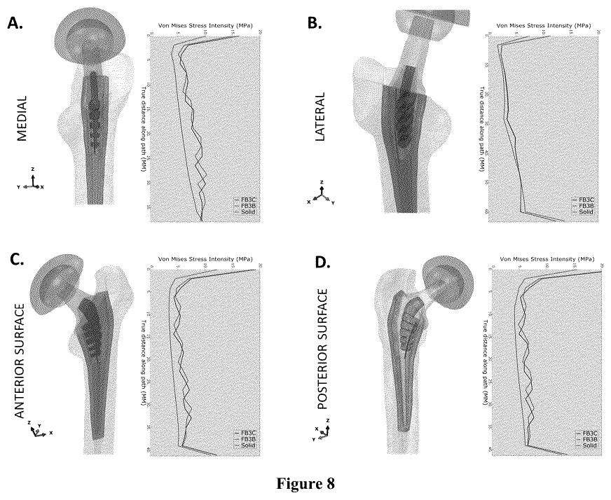

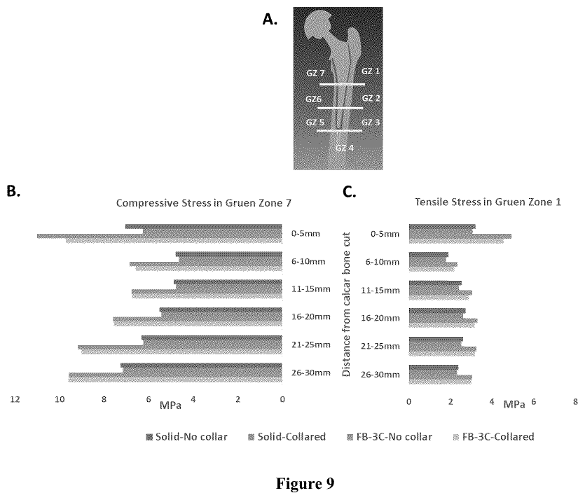

Stresses in proximal bone along the reamed bone cavity were evaluated with implants in interference fit. The minimization of stress shielding with truss implants extended through the proximal part of the implant. The pattern of von Mises stress was similar for both truss designs, FB-3B and FB-3C (FIG. 8). The difference in stress between the solid and truss designs was most significant on medial, anterior, and posterior bone interfaces (FIG. 8A,C,D). A minimal increase in stress was observed on the lateral interface was observed between the truss and solid designs (FIG. 8B). Averaging node values along the medial and lateral internal paths for every 5 mm segment in the cranial-caudal direction showed, as anticipated, peri-implant proximal bone to be under compressive stress on the medial side, and under tensile stress on the lateral side. The compressive stresses in medial bone were higher across all segments, more significantly in Gruen zone 7 with the truss implants compared to the solid configuration irrespective of the stem being collared or collarless (FIG. 9B). The maximal principal stress (tensile) in Gruen zone 1 was higher with truss implants, and the increase more noticeable in the most proximal segment (FIG. 9C). The presence of a calcar collar had no significant impact on compressive or tensile stress distribution in bone. The FB-3B and FB-3C stem designs with calcar collars showed comparable stress distribution in proximal bone on medial and lateral sides (Supplementary Figure S7A,B).

The medial-caudal flexure of the condylar neck increases compressive stresses on the medial side of the femoral neck and the diaphysis and tensile stresses at the femoral neck adjoining the greater trochanter (FIG. 3B). The bio-mimic flexure of the truss implant creates stress patterns similar to the natural femur, and the tensile stress, in order of 5 MPa, generated at the proximal lateral junction is well within the tensile yield strength of bone (51-66 MPa) [37].

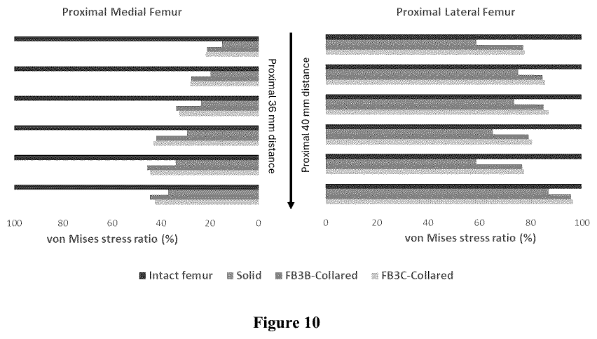

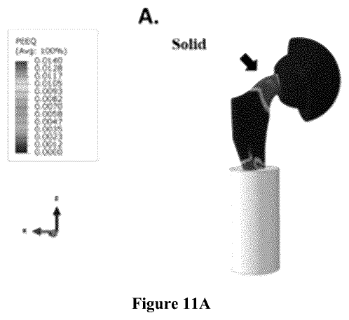

To determine the degree of stress shielding in relation to the intact femur, stress values in trabecular bone at implant interfaces and at corresponding nodes in the intact femur were used to calculate the von Mises stress ratio. Both truss stems showed higher stress ratios across Gruen zones 1 and 7 and they were more effective at reducing stress shielding than the solid stem (FIG. 10A,B). The stress in proximal lateral bone generated by FB-3B, FB-3C, and solid stems averaged 83%, 84%, and 70% of natural bone values, respectively (FIG. 10B). Increased medial calcar and cortical bone loading occurs with an engaged collar, and noticeably, stress in proximal medial trabecular bone adjacent to any stem type was markedly lower than intact bone (FIG. 10A). Average stress values in Gruen zone 7 were 26%, 36%, and 35% of intact bone for the solid and the FB-3B and FB-3C truss designs, respectively.

FIG. 8 shows von Misses stress intensity in proximal femur. von Misses stress values plotted in cranial-caudal direction at the (A) medial, (B) lateral, (C) anterior, and (D) posterior interference face. Line traces of solid, FB-3B, and FB-3C implants are overlayed in the graphs.

FIG. 9. Stress intensity in a proximal femur implanted with collared and collarless stems of solid and truss configurations. (A) The Gruen reference zones. Nodal stress values were averaged across 5 mm segments in cranial-caudal direction of the reamed bone cavity. (B) Minimum principal stress (compressive) in medial bone (Gruen zone 7), and (C) maximum principal stress (tensile) in proximal lateral bone (Gruen zone 1).

FIG. 10. von Mises stress ratio in proximal femur. The percentage change in stress was calculated by using stress intensity averaged across equally spaced proximal distance at implant interfaces of solid, collared FB-3B, and collared FB-3C implants and, correspondingly, in the intact femur. Stress ratio in (A) proximal medial femur (Gruen zone 7) and (B) proximal lateral femur (Gruen zone 1).

### 3.5. Implant Static Load Tests to Failure

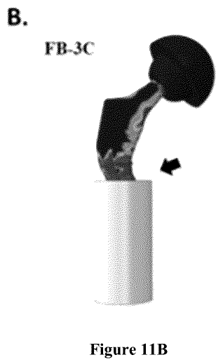

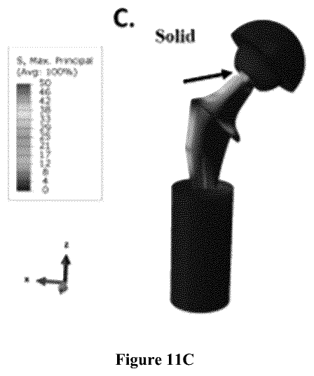

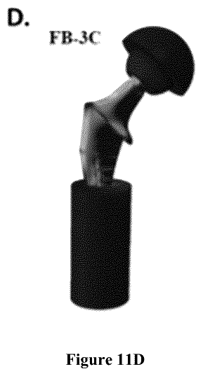

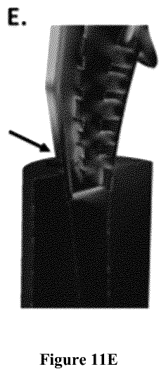

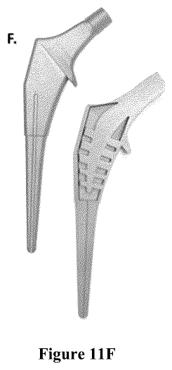

The distal stems of the implants were potted in the test cylinder and loaded similarly to previous ISO 7206-4:2010 tests, but herein, in a displacement-controlled manner until failure [34]. When the conjoined unit of the acetabular cup and the implant head were forced in the vertical direction in the local coordinate system for 50 mm, failure of the solid implant occurred at 85 kN due to neck gross yielding, while truss implants, FB-3B and FB-3C, failed at 63 kN due to local buckling of the medial implant wall near the potting level (FIG. 11A,B). Based on the plastic creep analysis, both implants were deemed suitably strong as the failure load exceeded the 2300 N test load in ISO standard by >25 fold.

FIG. 11 (A-E) shows endurance testing with ISO standard set-up. (A,B) Static load tests of solid and FB-3C implants. Gross yielding of neck region in solid implant and buckling of outer casing below trusses in the FB-3C implant (block arrows). Equivalent plastic strain (PEEQ) is set between 0 and 0.0140. (C,D) Maximum principal stresses in endurance tests of solid and FB-3C implants. Maximum principal stress is set between 0 and 50 MPa. (E) Expanded sectional view of FB-3C implant. Region of peak tensile stress at condylar head-neck junction in solid implant and at lower trusses in FB-3C implant (arrows). (F) Additively manufactured Ti-A16-4V truss implant, FB-3B, using laser powder bed fusion-external and vertically sectioned views are shown.

Embodiments of the femoral stem implant described herein focuses on selective debulking of the femoral stem to promote transfer of loads from implant to femur. The femoral stem concept has an outer shell and an internal structure that incorporates strategically introduced voids or hollowed-out regions. The biomechanical response of the implant occurs as a result of the controlled deformation accommodation of the outer shell. Loading of the implant causes the outer shell to reversibly stretch/deform/buckle resulting in the transfer of compressive and tensile stresses to the bone.

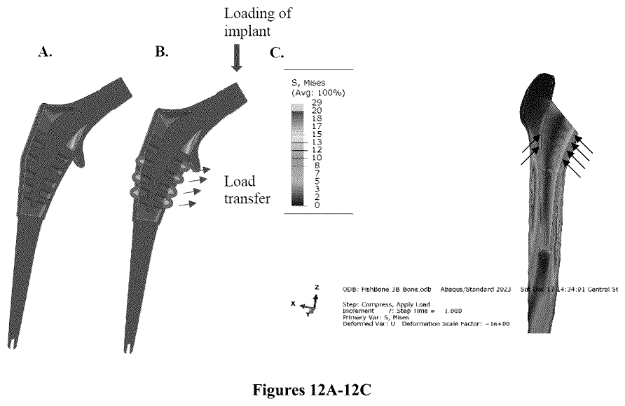

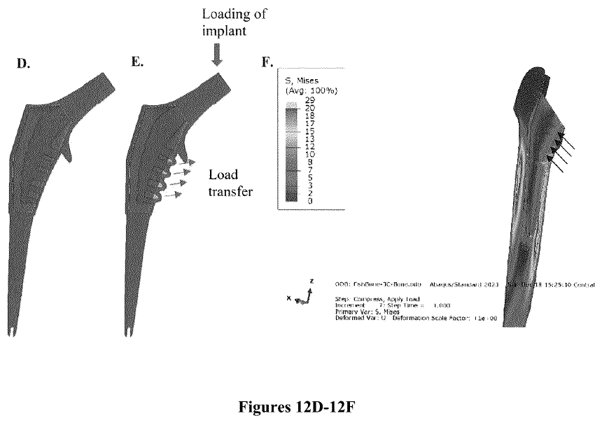

FIGS. 12A-12F show two examples of hip implant designs (A, D) with specific placement of internal hollowed areas that promotes flexible deformation of the implant wall/outer shell. The reversible controlled deformation of the exterior wall of the implant when loads are applied results in load transfer to abutting bone (B, E; red arrows). The transfer of compressive and tensile stresses in bone is observed in Von Mises stress plots of femur (C, F; black arrows).

### 3.6. Implant Endurance Load Tests

The implant wall, central spine, and trusses of FB-3C were joined together in 3D CAD with fillets added through the inner implant cavity. The stresses are mesh sensitive and based on earlier experience, a mesh size of 0.05 to 0.10 mm at critical locations in fatigue models was used to provide acceptable peak stress results. On cyclic loading of 2300 N to assess endurance properties [33], the solid implant displayed peak tensile stress of 65 MPa at the superior surface of the condylar head-to-neck transition (FIG. 11C). As the loading was unidirectional (not reverse bending), the alternating stress amplitude was computed as 33 MPa. Despite a relatively high-end single K-factor of 3.0, the stress of ˜100 MPa was determined to be well below the limit of alternating stress amplitude of 600 MPa to reach 10 million cycles.

Due to the increased flexibility of the FB-3C truss implant, the transition region at the neck had lower peak stress (FIG. 11D). As the loading was strongly focused on the lower distal portion of the implant that was immobilized, the lower trusses of the skeletal framework showed a higher peak stress of 96 MPa (FIG. 11E). This stress was not present in previous tests in implants placed in the femur as the loading set-up was different. When embedded in the femur, much of the load was shouldered by the proximal abutting bone. Being potted low on the distal stem for ISO endurance testing, a greater load was placed on the part of the implant not expected to bear such load. Nonetheless, endurance calculations using a highly conservative single K factor of 5.0 to account for unknowns in fabrication technique, an alternating stress amplitude of 240 MPa was calculated. With a 10 million cycle endurance limit of 600 MPa, the stress state of the truss implant was considered low enough to achieve the desired 10 million cycles of loading.

### 4. DISCUSSION

The aim of the study was to increase the load sharing capability of the femoral stem in the femur. Iterative debulked configurations were designed to lower the stiffness of the proximal stem and evaluated for load sharing and fatigue behavior by finite element analysis. The stems had a truss framework in the proximal part that was encased within the implant wall—a continuous surface that allows bone attachment and contact loading.

Aseptic loosening of implants is the most common reason for hip revision surgeries [38,39]. Considerable stress shielding in femurs implanted with solid stems results in significant bone loss, especially in the proximal medial region, Gruen zone 7, and less distally, in Gruen zones 3-5 [40]. Previous stem modifications to overcome the paucity in stress sharing had limited success [10-12,14-16,22,41,42]. As peri-implant bone loss invariably begins in the proximal femur, the rationale for the new design was to impart contextual flexibility to the proximal stem to promote load sharing and support bone mineral density for long-term implant stability. A debulking effort to return stresses to the bone was evaluated. The static strength of any debulked material is inherently weaker than a solid configuration of the same cross-sectional design. However, the internal truss design effectively countered the weaknesses of the hollow design while minimizing stress shielding in proximal bone. In interference fit, implant designs FB-3B and FB-3C demonstrated the best combination of structural rigidity, selective flexibility, and load sharing characteristics. Both designs had lower stresses in the implant than the solid form, afforded by greater load transfer to the femur due to their flexible distribution. With lowest implant stresses in the hemi-truss frame of FB-3C, the study focused on assessing the effect of the calcar collar and of cement fixation on performance of this stem. Load-adaptive stems that simulate the mechanical behavior of the natural femur create compressive stresses in medial proximal bone and in corollary, tensile stresses at the superior surface of the femoral neck and the lateral aspect of the greater trochanter. There were two salient observations that emerged from our study. First, the poor tensile strength of cement could increase the potential for creation of cracks in the material, leading to premature loss of implants with load-adaptive designs. Second, the external implant feature of a calcar collar influences stress within the implant. Evidence suggests that a calcar-collar contact fit promotes calcar loading and stem stability against rotational and torsional forces [43,44]. Although proximal medial trabecular bone in implanted femur was significantly underloaded compared to intact femur, collared stems, in previous experimental work, have shown superior strain distribution in the proximal region than collarless stems [43]. The calcar collar in the truss implant is critical to reducing implant stress and is suggested to improve strain distribution in cortical bone as well as protect the truss framework against torsional loads. For an extended service life, femoral stems with decreased proximal rigidity benefit from a collared stem that is press-fitted in bone.

The effect of mechanical strain in directing the fate of mesenchymal stem cells to cells of osteogenic lineage has been demonstrated in previous studies [45-47]. Differentiation of stem cells to osteoblasts promotes the deposition of new bone, and it is conceivable that mechanical stimulation through controlled elastic deformation of the implant wall will increase bone formation and truss implant osseointegration. Mechanical loading is vital to bone homeostasis and by fostering implant-bone contact and, by improving load transfer, the truss implant will limit the osteolysis of stress shielding.

Rehabilitation regimens after hip replacements that favor a delay or a reduction in weight bearing can accentuate bone deterioration [48]. Although the type of weight bearing regimen following hip implantation lacks consensus [48-50], there is a general agreement on the benefits of early loading to preserving bone mineral density. Rigid implants have a propensity for stress shielding, and without a perfect fit between the implant and the reamed femoral canal, rigid stems generate hard and light contact zones which promote unequal loading of bone and osteolysis over time. A flexible implant has the benefit of an improved interference fit that promotes consistent contact loading and a more uniform distribution of stresses. Although the effect of interference fit was not tested in the current work, a reduction in Young's modulus generated through the debulking effort will create a compressive stress field at the proximal bone interface, reducing the detrimental effects of tensile stresses in bone and facilitating the early initiation of weight bearing.

Additive manufacturing (AM) provides an opportunity to construct structures of complex geometries that cannot be made using subtractive manufacturing processes. With the advent of AM, the construction of metal 3D lattices and truss designs for medical applications has become feasible. Another advantage of the use of AM in these applications is the capability to produce patient-specific, individualized implants and devices that match the patient's anatomy and activity level. As the stiffness of a metallic implants is several times higher than that of the surrounding bone, porous and auxetic designs have been studied to achieve bone-equivalent elasticity in an implanted material with the added benefit of improved osseointegration. Although agreement is lacking regarding the pore size for optimal bone growth, studies suggest that pores >300 μm support new bone formation and neovascularization [51,52]. Highly porous structures with a pore size of 700 μm and a porosity of 70-90% greatly facilitate bone growth and are ideal bone scaffolds [53], and completely porous designs in this respect have the inherent advantage of facilitating bone ingrowth for implant anchorage. However, other than the limitation of mechanical strength, the extent of bone invagination and calcification can affect the mechanical situation and the predicted performance of the implant. In this regard, an internal truss framework that is compartmentalized from external influences has the advantage of a predictable and consistent performance.

Assessment of bone mineral density through long-term radiological examination of femurs with porous implants suggests bone decalcification besides porous regions and spot-welds of dense bone being common distal to the porous area. The loss of bone density was most significant in Gruen zone 7 irrespective of the two porous designs [54]. An ex vivo assessment of a stem of graded porosity, sheathed within a smooth shell on the medial and lateral sides, effectively limited stress shielding, most prominently in Gruen zones more distal to the porous structure [15]. In contrast, our work demonstrates that the truss implant best improves loading at the proximal bone. The fatigue study as per ISO standards simulates the worst-case scenario in which the proximal femur is totally resorbed, and the bone support is limited to the distal portion of the stem. It does not truly replicate a situation where the femoral stem is proximally well-fixed. Similar to a prior study, stress concentration at the potting junction along the medial implant wall was observed in our study as well [15]. Though the peak stress in endurance testing occurred at the most distal truss, the truss implant in our analysis exceeded ISO requirements and was considered suitable in strength for expected loads acting on the body during normal activity.

Mechanical properties of additively manufactured parts with porous geometries depend on the type of unit cell, the degree of microscaling of the unit, and its relative density [555]. Fatigue cracks often originate from material irregularities or geometric defects, which are areas of higher stresses [56]. The fatigue behavior of pure titanium and titanium alloy auxetic structures characteristically begin with a large initial deformation followed by densification and multiple strain jumps that correspond to layer-on-layer collapse [57,58]. In general, fatigue performance is lower for auxetic materials compared to materials of positive Poisson's ratio, but it is argued that the combination of the two materials could be utilized to achieving the desired mechanical characteristics in orthopedic implants [26,-57]. As auxetic structures contract laterally under compression while expand under tension, their incorporation could be beneficial in stretch-dominated areas such as the proximal lateral femoral stem but have limited effectiveness on the medial side where large compressive forces are present.

With the introduction of AM, customized prototype printing of medical devices, instruments, and parts with distinctive properties and added functionality are feasible. However, geometric parameters (such as strut diameter and length) and manufacturing inclination influence material performance and structural efficiency. The slenderness ratio (strut diameter:strut length) affects the critical buckling load, and struts of small ratios are considerably less resilient to buckling [-59]. Based on the angle the struts form to the powder bed platform, the build orientation angle is another parameter that affects the quality of struts. Struts at low orientation angles are low in quality; horizontal beams are of lowest quality [60]. Horizontal, micro-scaled struts in lattice and auxetic designs can be vulnerable to early failure. In addition, defects introduced during the additive manufacturing process can impact material behavior. Gas porosities, partial melt pools, and surface roughness are common imperfections in powder bed fusion fabrication [61-62]. The two major contributors that lower the physical properties of metal structures are surface roughness and subsurface pores. Surface-connected porosities are often points of crack initiation and cracks that propagate from the surface into the material can lead to structural failure. Altering build orientation, and post-processing HIP and surface treatments can reduce imperfections and improve service life [61,63,64], but in general, the presence of defects in thin structures can be critical and significantly life-limiting. Debris from cyclic loading of flexible structures can cause inflammation and negatively impact bone integrity. In this respect, the proposed design that encases beam-like thick trusses within the implant alleviates the concern, however, their inaccessibility to surface treatments could be a factor that impacts fatigue life. Aggressive vibration before releasing the metal powder from the cavity could produce a deburring and shot peening effect to reduce surface irregularities. However, the efficiency of this process in sufficiently reducing surface and subsurface flaws is uncertain. The imperfections in AM fabrication and inhomogeneity of the microstructure necessitate the understanding of fatigue behavior of AM material, an element critical to the success of the truss implant.

Sites of high tensile stress invariably initiate fatigue cracks and notch positions are particularly vulnerable [56]. Truss and implant wall fatigue could be limitations of the design, and by iteratively designing out stress raisers and moving high general stress areas away from the discontinuity features could reduce fatigue failures. To this end, the optimization of the hemi-truss design to drop the peak stresses at distal truss could be achieved through simple nonintrusive geometry changes. The alternative design with the balanced truss framework affords greater rigidity and mechanical strength against torsional loads, and a comparative evaluation of the hemi-truss and balanced-truss framework needs additional studies. Non-modular and modular stems of varying length, taper, neck length, and neck angle are in clinical use, and the high tuning of the truss design in individual contexts is merited. Lastly, as the interference fit creates a compressive field on the truss implant, the effects on implant-bone contact need evaluation.

### 5. CONCLUSIONS

Our main finding is that with a truss implant, stability is achieved through a compressive stress field. We suggest that this field will support early weight bearing and the deformation of the implant wall will stimulate new bone formation. Concurrently, stress shielding will be reduced, and fatigue life will be maintained. Of the five models considered in our study, the truss implant model FB-3C showed the best results, especially in presenting a much-reduced stress shielding effect compared to the solid implant model. Notably, our results suggest that the use of cement will be less efficient than press-fit fixation for long-term stability of the truss implant.

### REFERENCES

1. Pivec, R.; Johnson, A. J.; Mears, S. C.; Mont, M. A. Hip arthroplasty. Lancet 2012, 380, 1768-1777.
2. Goldman, A. H.; Osmon, D. R.; Hanssen, A. D.; Pagnano, M. W.; Berry, D. J.; Abdel, M. P. The Lawrence D. Dorr Surgical Techniques & Technologies Award: Aseptic Reoperations Within One Year of Primary Total Hip Arthroplasty Markedly Increase the Risk of Later Periprosthetic Joint Infection. J. Arthroplast. 2020, 35, S10-S14.
3. Ulrich, S. D.; Seyler, T. M.; Bennett, D.; Delanois, R. E.; Saleh, K. J.; Thongtrangan, I.; Kuskowski, M.; Cheng, E. Y.; Sharkey, P. F.; Parvizi, J.; et al. Total hip arthroplasties: What are the reasons for revision? Int. Orthop. 2008, 32, 597-604.
4. Arabnejad Khanoki, S.; Pasini, D. Multiscale design and multiobjective optimization of orthopedic hip implants with functionally graded cellular material. J. Biomech. Eng. 2012, 134, 031004.
5. Sumner, D. R.; Turner, T. M.; Igloria, R.; Urban, R. M.; Galante, J. O. Functional adaptation and ingrowth of bone vary as a function of hip implant stiffness. J. Biomech. 1998, 31, 909-917.
6. Feng, X.; Gu, J.; Zhou, Y. Primary total hip arthroplasty failure: Aseptic loosening remains the most common cause of revision. Am. J. Transl. Res. 2022, 14, 7080-7089.
7. Bieger, R.; Ignatius, A.; Decking, R.; Claes, L.; Reichel, H.; Durselen, L. Primary stability and strain distribution of cementless hip stems as a function of implant design. Clin. Biomech. 2012, 27, 158-164.
8. Hnat, W. P.; Conway, J. S.; Malkani, A. L.; Yakkanti, M. R.; Voor, M. J. The effect of modular tapered fluted stems on proximal stress shielding in the human femur. J. Arthroplast. 2009, 24, 957-962.
9. Jeon, I.; Bae, J. Y.; Park, J. H.; Yoon, T. R.; Todo, M.; Mawatari, M.; Hotokebuchi, T. The biomechanical effect of the collar of a femoral stem on total hip arthroplasty. Comput. Methods Biomech. Biomed. Engin 2011, 14, 103-112.
10. Heyland, M.; Checa, S.; Kendoff, D.; Duda, G. N. Anatomic grooved stem mitigates strain shielding compared to established total hip arthroplasty stem designs in finite-element models. Sci. Rep. 2019, 9, 482.
11. Arabnejad, S.; Johnston, B.; Tanzer, M.; Pasini, D. Fully porous 3D printed titanium femoral stem to reduce stress-shielding following total hip arthroplasty. J. Orthop. Res. 2017, 35, 1774-1783.
12. Limmahakhun, S.; Oloyede, A.; Chantarapanich, N.; Jiamwatthanachai, P.; Sitthiseripratip, K.; Xiao, Y.; Yan, C. Alternative designs of load-sharing cobalt chromium graded femoral stems. Mater. Today Commun. 2017, 12, 1-10. https://doi.org/10.1016/j.mtcomm.2017.05.002.
13. Wang, S.; Zhou, X.; Liu, L.; Shi, Z.; Hao, Y. On the design and properties of porous femoral stems with adjustable stiffness gradient. Med. Eng. Phys. 2020, 81, 30-38.
14. Gao, X.; Zhao, Y.; Wang, M.; Liu, Z.; Liu, C. Parametric Design of Hip Implant with Gradient Porous Structure. Front. Bioeng. Biotechnol. 2022, 10, 850184.
15. Naghavi, S. A.; Tamaddon, M.; Garcia-Souto, P.; Moazen, M.; Taylor, S.; Hua, J.; Liu, C. A novel hybrid design and modelling of a customised graded Ti-6Al-4V porous hip implant to reduce stress-shielding: An experimental and numerical analysis. Front. Bioeng. Biotechnol. 2023, 11, 1092361.
16. Liu, B.; Wang, H.; Zhang, N.; Zhang, M.; Cheng, C. K. Femoral Stems with Porous Lattice Structures: A Review. Front. Bioeng. Biotechnol. 2021, 9, 772539.
17. Huiskes, R. I. K.; Weinans, H.; Rietbergen, B. V. The Relationship Between Stress Shielding and Bone Resorption Around Total Hip Stems and the Effects of Flexible Materials. Clin. Orthop. Relat. Res. 1992, 274, 124-134.
18. Adam, F.; Hammer, D. S.; Pfautsch, S.; Westermann, K. Early failure of a press-fit carbon fiber hip prosthesis with a smooth surface. J. Arthroplast. 2002, 17, 217-223.
19. Trebse, R.; Milosev, I.; Kovac, S.; Mikek, M.; Pisot, V. Poor results from the isoelastic total hip replacement: 14-17-year follow-up of 149 cementless prostheses. Acta Orthop. 2005, 76, 169-176.
20. Hanada, S.; Masahashi, N.; Jung, T. K.; Yamada, N.; Yamako, G.; Itoi, E. Fabrication of a high-performance hip prosthetic stem using beta Ti-33.6Nb-4Sn. J. Mech. Behav. Biomed. Mater. 2014, 30, 140-149.
21. Chiba, D.; Yamada, N.; Mori, Y.; Oyama, M.; Ohtsu, S.; Kuwahara, Y.; Baba, K.; Tanaka, H.; Aizawa, T.; Hanada, S.; et al. Mid-term results of a new femoral prosthesis using Ti—Nb—Sn alloy with low Young's modulus. BMC Musculoskelet. Disord. 2021, 22, 987.
22. Yamako, G.; Janssen, D.; Hanada, S.; Anijs, T.; Ochiai, K.; Totoribe, K.; Chosa, E.; Verdonschot, N. Improving stress shielding following total hip arthroplasty by using a femoral stem made of beta type Ti-33.6Nb-4Sn with a Young's modulus gradation. J. Biomech. 2017, 63, 135-143.
23. Wang, R.; Ni, S.; Ma, L.; Li, M. Porous construction and surface modification of titanium-based materials for osteogenesis: A review. Front. Bioeng. Biotechnol. 2022, 10, 973297.
24. Zadpoor, A. A. Mechanical performance of additively manufactured meta-biomaterials. Acta Biomater. 2019, 85, 41-59.
25. Kolken, H. M. A.; de Jonge, C. P.; van der Sloten, T.; Garcia, A. F.; Pouran, B.; Willemsen, K.; Weinans, H.; Zadpoor, A. A. Additively manufactured space-filling meta-implants. Acta Biomater. 2021, 125, 345-357.
26. Kolken, H. M. A.; Janbaz, S.; Leeflang, S. M. A.; Lietaert, K.; Weinans, H. H.; Zadpoor, A. A. Rationally designed meta-implants: A combination of auxetic and conventional meta-biomaterials. Mater. Horiz. 2018, 5, 28-35.
27. Liu, B.; Wang, H.; Zhang, M.; Li, J.; Zhang, N.; Luan, Y.; Fang, C.; Cheng, C. K. Capability of auxetic femoral stems to reduce stress shielding after total hip arthroplasty. J. Orthop. Translat 2023, 38, 220-228.
28. Rana, M.; Karmakar, S.; Bandyopadhyay, A.; Roychowdhury, A. Design and manufacturing of patient-specific Ti6Al4V implants with inhomogeneous porosity. J. Mech. Behav. Biomed. Mater. 2023, 143, 105925.
29. Morgan, E. F.; Bayraktar, H. H.; Keaveny, T. M. Trabecular bone modulus-density relationships depend on anatomic site. J. Biomech. 2003, 36, 897-904.
30. Javid, S. Optimized Evaluation of Bone Tissue Material Properties by Inverse Finite Element Analysis and Femur Fracture Testing. Ph.D. Dissertation, North Dakota State University of Agriculture and Applied Science, Fargo, ND, USA, October 2014.
31. Lampman, S. R. ASM Handbook. Fatigue and Fracture; ASM International: Almere, The Netherlands, 1996; Volume 19, p. 842.
32. ISO Standard No. 7206-4:2010; Implants for Surgery—Partial and Total Hip Joint Prostheses—Part 4: Determination of Endurance Properties and Performance of Stemmed Femoral Components. ISO, International Organization for Standardization: Geneva, Switzerland, 2010.
33. ISO Standard No. 7206-6:2013; Implants for Surgery—Partial and Total Hip Joint Prostheses—Part 6: Endurance Properties Testing and Performance Requirements of Neck Region of Stemmed Femoral Components. ISO, International Organization for Standardization: Geneva, Switzerland, 2013.
34. ISO Standard No. 7206-10:2018; Implants for Surgery—Partial and Total Hip Joint Prostheses—Part 10: Determination of Resistance to Static Load of Modular Femoral Heads. ISO, International Organization for Standardization: Geneva, Switzerland, 2018.
35. Bergmann, G.; Bender, A.; Dymke, J.; Duda, G.; Damm, P. Standardized Loads Acting in Hip Implants. PLOS ONE 2016, 11, e0155612.
36. Beckmann, N. A.; Bitsch, R. G.; Seeger, J. B.; Klotz, M. C.; Kretzer, J. P.; Jaeger, S. Mechanical properties of a cemented porous implant interface. Acta Orthop. 2014, 85, 531-537.
37. Kundu, J.; Pati, F.; Shim, J.-H.; Cho, D.-W. (Eds.) Rapid Prototyping Technology for Bone Regeneration. In Rapid Prototyping of Biomaterials—Principles and Applications; Narayan, R. Ed.; Woodhead Publishing: Sawston, UK, 2014.
38. AAOS. American Joint Replacement Registry (AJRR): 2022 Annual Report; AAOS: Rosemont, IL, USA, 2022.
39. Deere, K.; Whitehouse, M. R.; Kunutsor, S. K.; Sayers, A.; Mason, J.; Blom, A. W. How long do revised and multiply revised hip replacements last? A retrospective observational study of the National Joint Registry. Lancet Rheumatol. 2022, 4, e468-79.
40. Inaba, Y.; Kobayashi, N.; Oba, M.; Ike, H.; Kubota, S.; Saito, T. Difference in Postoperative Periprosthetic Bone Mineral Density Changes Between 3 Major Designs of Uncemented Stems: A 3-Year Follow-Up Study. J. Arthroplast. 2016, 31, 1836-1841.
41. Gross, S.; Abel, E. W. A finite element analysis of hollow stemmed hip prostheses as a means of reducing stress shielding of the femur. J. Biomech. 2001, 34, 995-1003.
42. Yang, C. T.; Wei, H. W.; Kao, H. C.; Cheng, C. K. Design and test of hip stem for medullary revascularization. Med. Eng. Phys. 2009, 31, 994-1001.
43. Johnson, A. J.; Desai, S.; Zhang, C. Y.; Koh, K.; Zhang, L. Q.; Costales, T.; O'Toole, R. V.; Manson, T. T. A calcar collar is protective against early torsional/spiral periprosthetic femoral fracture: A paired cadaveric biomechanical analysis. J. Bone Joint Surg. Am. 2020, 102, 1427-1433.
44. Levadnyi, I.; Gubaua, J. E.; Dicati, G. W. O.; Awrejcewicz, J.; Gu, Y. D.; Pereira, J. T.; Loskutov, A. Comparative analysis of the biomechanical behavior of collar and collarless stems: Experimental testing and finite element modelling. J. Med. Biol. Eng. 2021, 41, 844-855.
45. Chang, Y.; Shao, Y.; Liu, Y.; Xia, R.; Tong, Z.; Zhang, J.; Zhai, Z.; Cheng, W.; Li, H. Mechanical strain promotes osteogenic differentiation of mesenchymal stem cells on TiO(2) nanotubes substrate. Biochem. Biophys. Res. Commun. 2019, 511, 840-846.
46. Liu, Y.; Cheng, W.; Zhao, Y.; Gao, L.; Chang, Y.; Tong, Z.; Li, H.; Jing, J. Cyclic Mechanical Strain Regulates Osteoblastic Differentiation of Mesenchymal Stem Cells on TiO(2) Nanotubes Through GCN5 and Wnt/beta-Catenin. Front. Bioeng. Biotechnol. 2021, 9, 735949.
47. Wang, J.; Wang, C. D.; Zhang, N.; Tong, W. X.; Zhang, Y. F.; Shan, S. Z.; Zhang, X. L.; Li, Q. F. Mechanical stimulation orchestrates the osteogenic differentiation of human bone marrow stromal cells by regulating HDAC1. Cell Death Dis. 2016, 7, e2221.
48. Boden, H.; Adolphson, P. No adverse effects of early weight bearing after uncemented total hip arthroplasty: A randomized study of 20 patients. Acta Orthop. Scand. 2004, 75, 21-29.
49. Wolf, O.; Mattsson, P.; Milbrink, J.; Larsson, S.; Mallmin, H. Periprosthetic bone mineral density and fixation of the uncemented CLS stem related to different weight bearing regimes: A randomized study using DXA and RSA in 38 patients followed for 5 years. Acta Orthop. 2010, 81, 286-291.
50. Wolf, O.; Mattsson, P.; Milbrink, J.; Larsson, S.; Mallmin, H. Effects of postoperative weight-bearing on body composition and bone mineral density after uncemented total hip arthroplasty. J. Rehabil. Med. 2013, 45, 498-503.
51. Ran, Q.; Yang, W.; Hu, Y.; Shen, X.; Yu, Y.; Xiang, Y.; Cai, K. Osteogenesis of 3D printed porous Ti6Al4V implants with different pore sizes. J. Mech. Behav. Biomed. Mater. 2018, 84, 1-11. https://doi.org/10.1016/j.jmbbm.2018.04.010.
52. Chang, B.; Song, W.; Han, T.; Yan, J.; Li, F.; Zhao, L.; Kou, H.; Zhang, Y. Influence of pore size of porous titanium fabricated by vacuum diffusion bonding of titanium meshes on cell penetration and bone ingrowth. Acta Biomater. 2016, 33, 311-321.
53. Zhang, Y.; Sun, N.; Zhu, M.; Qiu, Q.; Zhao, P.; Zheng, C.; Bai, Q.; Zeng, Q.; Lu, T. The contribution of pore size and porosity of 3D printed porous titanium scaffolds to osteogenesis. Biomater. Adv. 2022, 133, 112651.
54. Matsuyama, K.; Ishidou, Y.; Guo, Y. M.; Kakoi, H.; Setoguchi, T.; Nagano, S.; Kawamura, I.; Maeda, S.; Komiya, S. Finite element analysis of cementless femoral stems based on mid- and long-term radiological evaluation. BMC Musculoskelet. Disord. 2016, 17, 397.
55. Zadpoor, A. A. Additively manufactured porous metallic biomaterials. J Mater Chem B. 2019, 7, 4088-4117.
56. Zerbst, U.; Madia, M.; Klinger, C.; Bettge, D.; Murakami, Y. Defects as a root cause of fatigue failure of metallic components. I: Basic aspects. Eng. Fail. Anal. 2019, 97, 777-792.
57. Kolken, H. M. A.; Garcia, A. F.; Du Plessis, A.; Rans, C.; Mirzaali, M. J.; Zadpoor, A. A. Fatigue performance of auxetic meta-biomaterials. Acta Biomater. 2021, 126, 511-523.
58. Kolken, H. M. A.; Garcia, A. F.; Plessis, A. D.; Meynen, A.; Rans, C.; Scheys, L.; Mirzaali, M. J.; Zadpoor, A. A. Mechanisms of fatigue crack initiation and propagation in auxetic meta-biomaterials. Acta Biomater. 2022, 138, 398-409.
59. Alghamdi, A.; Downing, D.; Tino, R.; Almalki, A.; Maconachie, T.; Lozanovski, B.; Brandt, M.; Qian, M.; Leary, M. Buckling phenomena in AM lattice strut elements: A design tool applied to Ti-6Al-4V LB-PBF. Mater. Des. 2021, 208, 109892.
60. Wauthle, R.; Vrancken, B.; Beynaerts, B.; Jorissen, K.; Schrooten, J.; Kruth, J. P.; Van Humbeeck, J. Effects of build orientation and heat treatment on the microstructure and mechanical properties of selective laser melted Ti6Al4V lattice structures. Addit. Manuf. 2015, 5, 77-84.
61. Afroz, L.; Das, R.; Qian, M.; Easton, M.; Brandt, M. Fatigue behaviour of laser powder bed fusion (L-PBF) Ti-6Al-4V, Al—Si—Mg and stainless steels: A brief overview. Int. J. Fract. 2022, 235, 3-46.
62. Segersall, M.; Kerwin, A.; Hardaker, A.; Kahlin, M.; Moverare, J. Fatigue response dependence of thickness measurement methods for additively manufactured E-PBF Ti-6Al4 V. Fatigue Fract. Eng. Mater. Struct. 2021, 44, 1931-1943.
63. Kahlin, M.; Ansell, H.; Basu, D.; Kerwin, A.; Newton, L.; Smith, B.; Moverare, J. J. Improved fatigue strength of additively manufactured Ti6Al4V by surface post processing. Int. J. Fatigue 2020, 134, 105497.
64. Kahlin, M.; Ansell, H.; Moverare, J. Fatigue crack growth for through and part-through cracks in additively manufactured Ti6Al4V. Int. J. Fatigue 2022, 155, 106608.

### Supplemental to Example 1

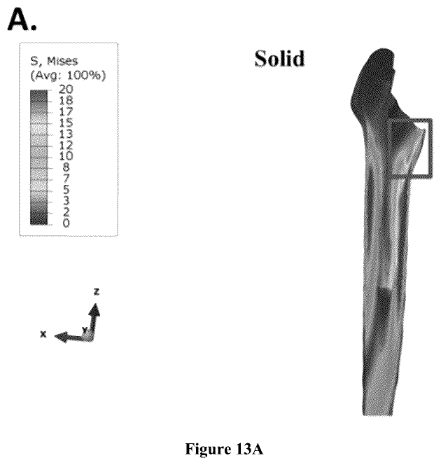

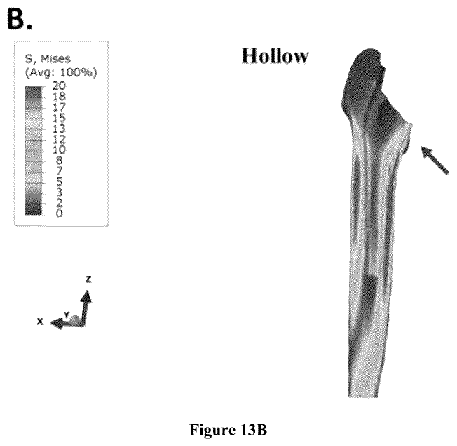

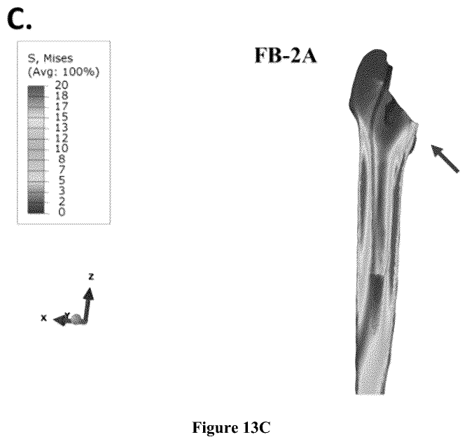

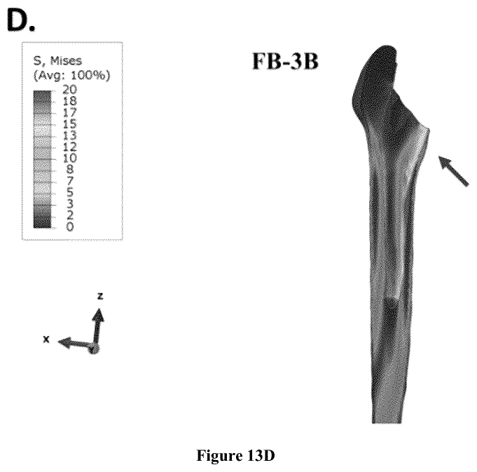

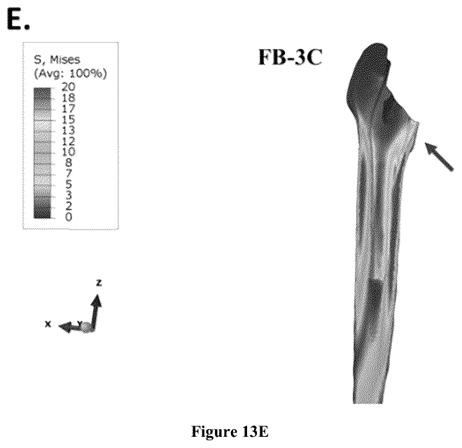

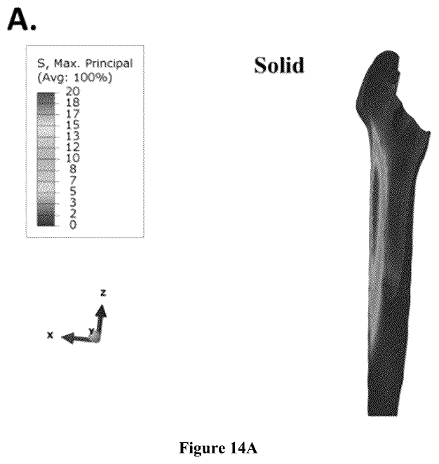

FIGS. 13A-13E show von Mises stress intensity in femur with cemented collared stems. (A) Solid, (B) hollow, (C) FB-2A, (D) FB-3B, and (E) FB-3C. Stress plots are set between 0-20 MPa for comparative purposes. Stress shielding in medial proximal bone (red frame). Increase in stress at proximal medial femur with debulked implants (arrows).

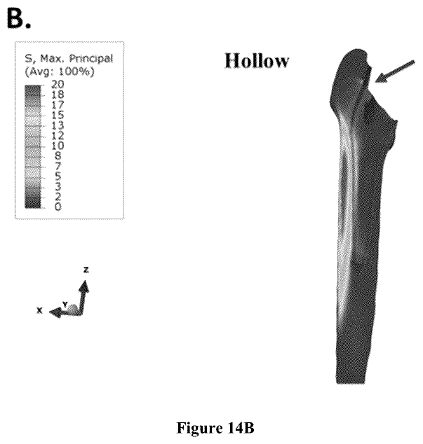

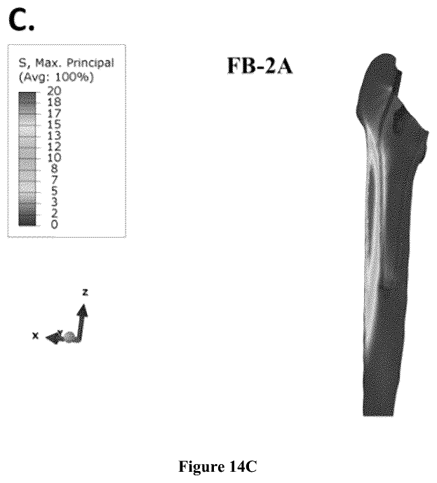

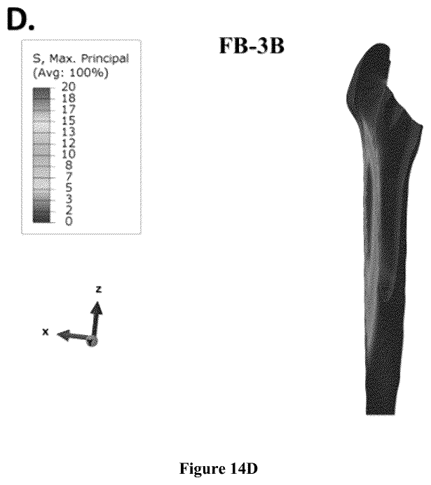

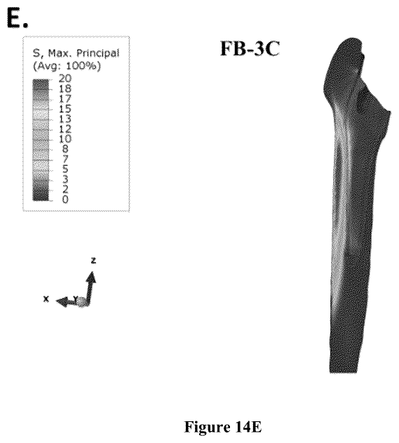

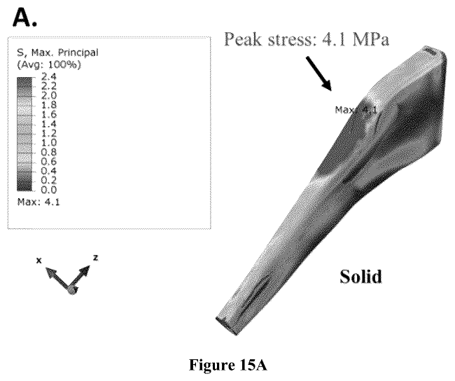

FIGS. 14A-14E show Maximum principal (tensile) stresses in femur with cemented collared stems. (A) Solid, (B) hollow, (C) FB-2A, (D) FB-3B, and (E) FB-3C. Stress plots are set between 0-20 MPa for comparative purposes. A localized increase in tensile stress at the proximal lateral connection between cement and femur (arrow).

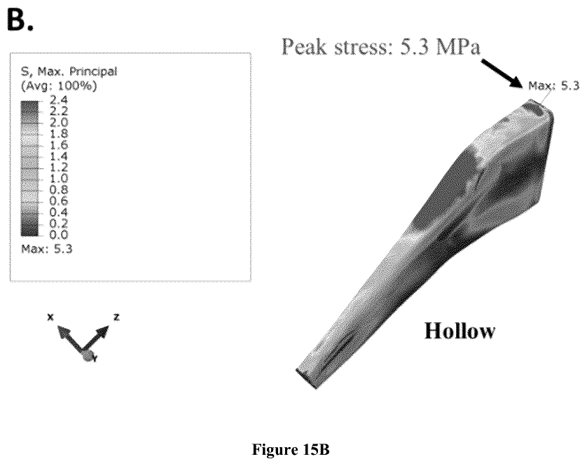

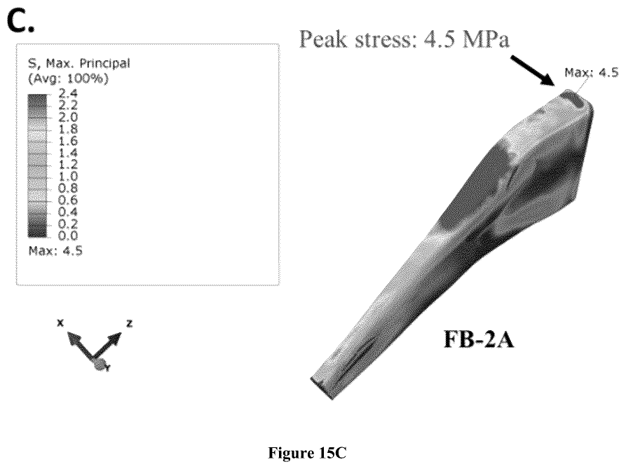

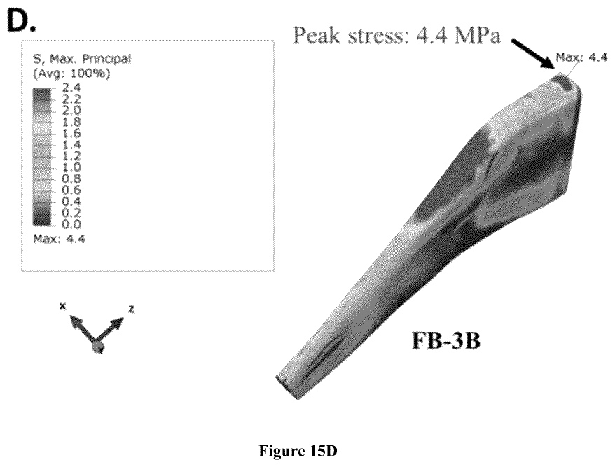

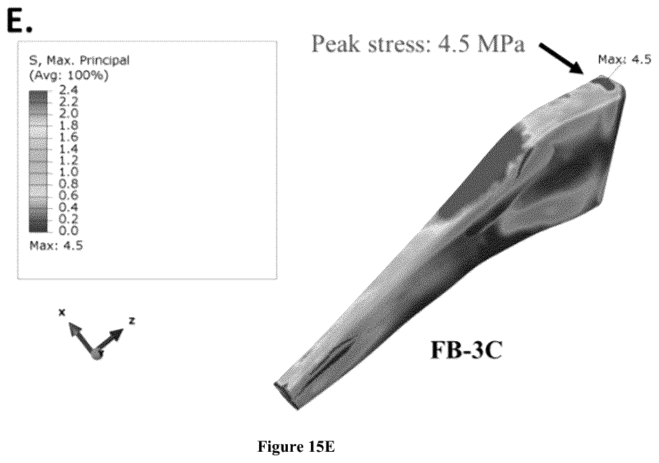

FIGS. 15A-15E show Maximum principal (tensile) stress at bone-cement interface with collared stems. A. Solid, B. hollow, C. FB-2A, D. FB-3B, E. FB-3C. Stress plots are set between 0-2.4 MPa for comparative purposes. Peak stress (arrows) with debulked implants occur at proximal lateral bone-cement interface.

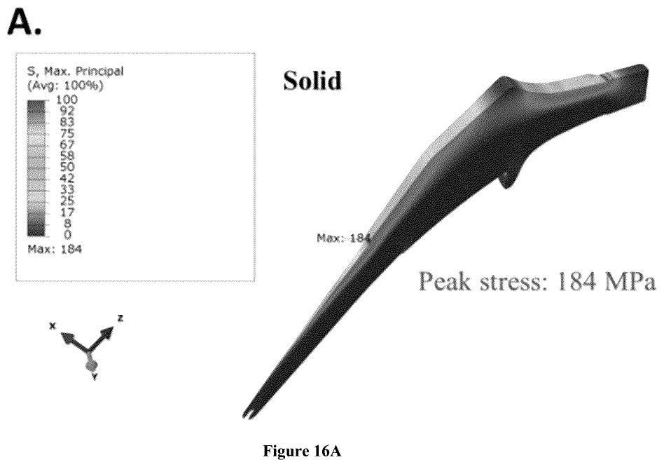

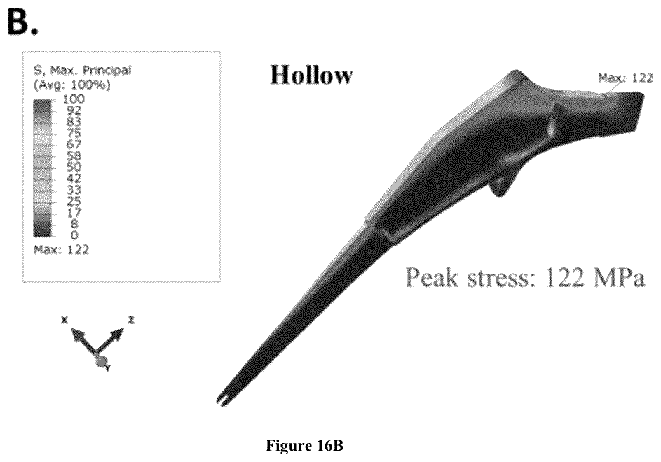

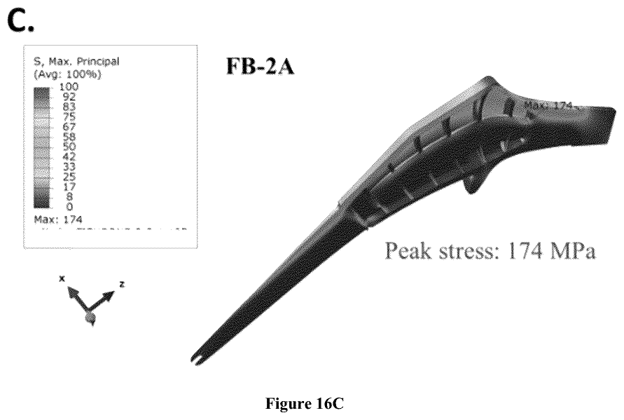

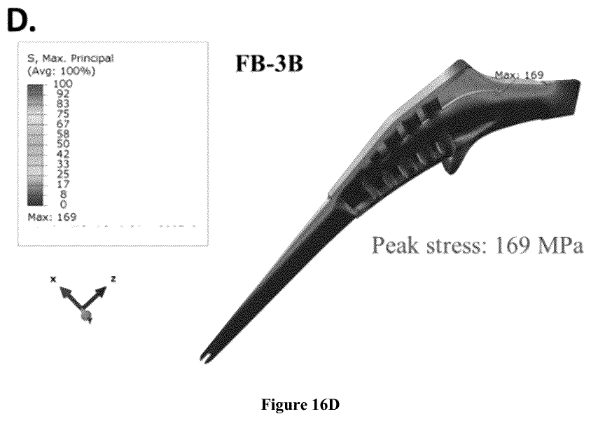

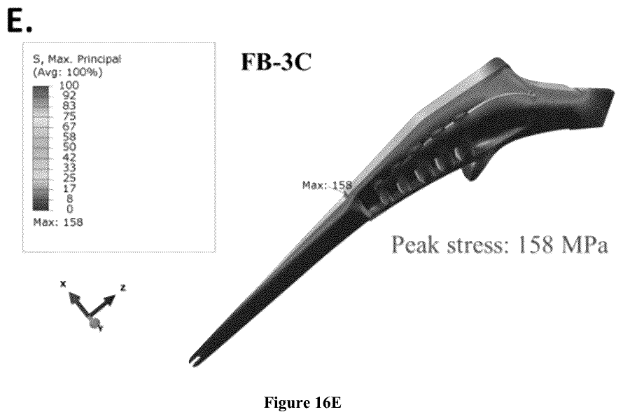

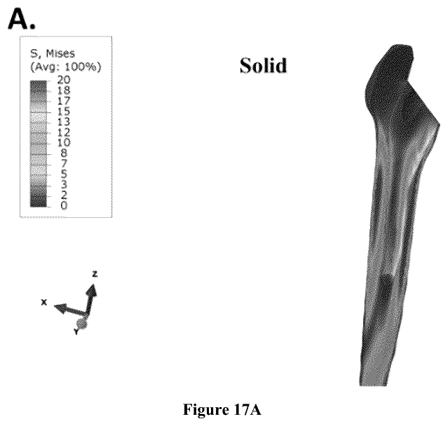

FIGS. 16A-16 show Maximum principal stress in collared stems when cemented. (A) Solid, (B) hollow, (C) FB-2A, (D) FB-3B, and (E) FB-3C. Stress plots are set between 0-100 MP a for comparative purposes.

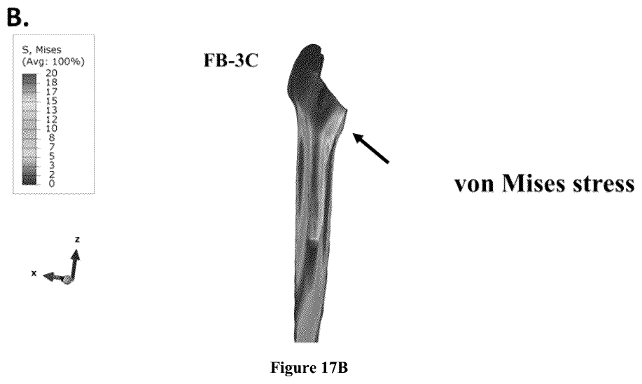

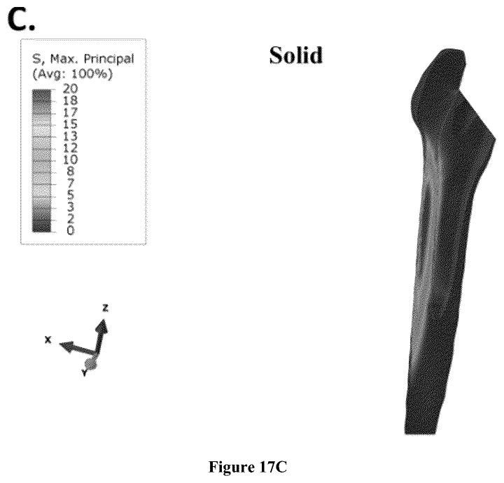

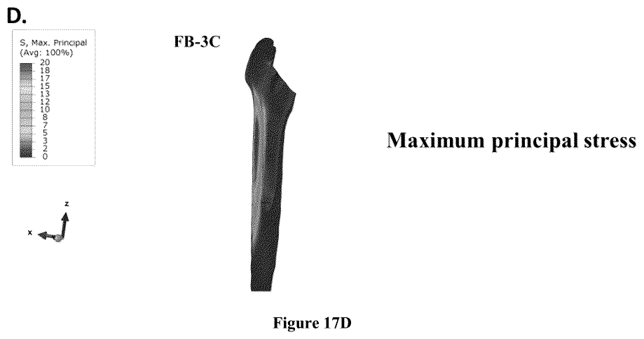

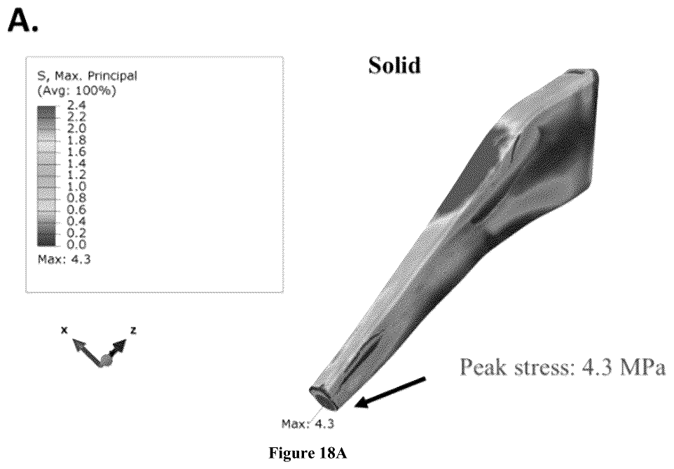

FIGS. 17A-17D show Stress in femur with cemented collarless stems. (A, B) von Mises stress and (C, D) maximum principal stress. (A, C) Solid implant. (B, D) FB-3C implant. Stress plots are set between 0-20 MPa for comparative purposes. Noticeably higher compressive stress at proximal medial bone with FB-3C implant than solid implant (arrow).

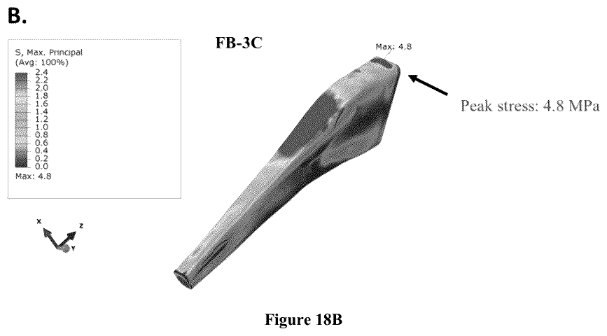

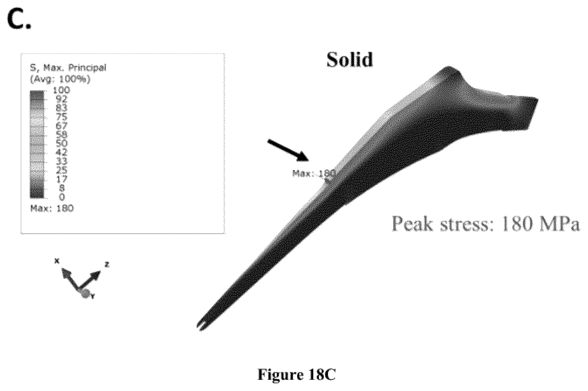

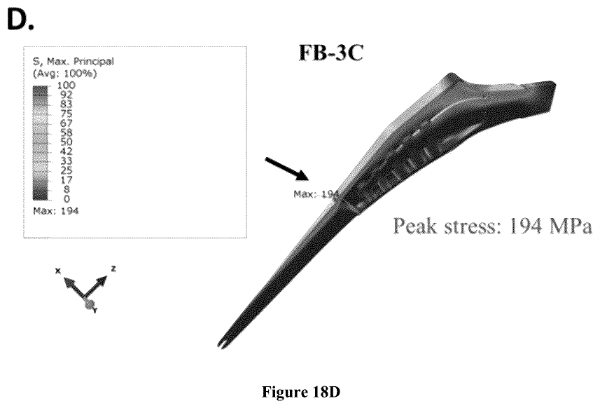

FIGS. 18A-18D show Maximum principal stress intensity in collarless stems and at cement-bone interface. (A,B) Tensile stresses at cement interface with solid implant and FB-3C implant. (C,D) Tensile stress in collarless solid and FB-3C implants. Peak stress (arrows) in collarless implants-solid: 180 MPa without collar compared to 182 with collar; FB-3C: 194 MPa without collar compared to 159 MPa with collar.

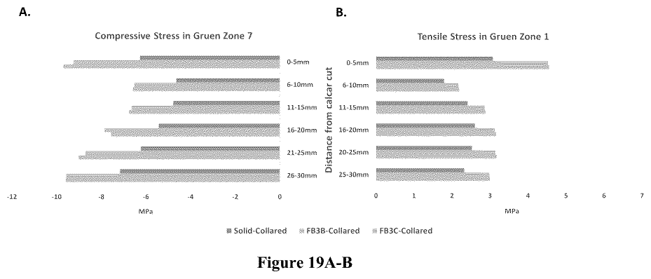

FIG. 19A-B show Compressive and tensile stress in proximal femur implanted with collared solid, FB-3B, and FB-3C stems. Nodal stress values were averaged across 5 mm segments in cranial-caudal direction of the reamed bone cavity. (A) Minimum principal stress (compressive) in medial bone (Gruen zone 7), and (B) maximum principal stress (tensile) in proximal lateral bone (Gruen zone 1).

### Example 2

There are multiple advantages for making a femoral implant more flexible, from a more consistent interference contact layer between the bone and the implant and easier installation, to reducing stress shielding and generally better stress distribution from the implant to the femur. The effort of this study is to investigate optimal methods for debulking the femoral implant yet retain sufficient strength and endurance properties. The debulking effort herein focuses on the use of an internal fishbone skeletal structure with a 2-mm outer casing. The implant is solid from the head to the medial collar, and then again at the distal region of the stem. Various configurations were created and tested with finite element analysis (PEA) using ISO 7206 loads, both with the implant in a natural femur and in the typical testing setup outlined by ISO 7206. Ultimately, a fishbone structure was devised with no trusses located above the medial collar and minimal trusses on the lateral side of the implant spine/central post. The trusses were thickened and flattened to provide greater strength. This design promoted the best combination of sufficient rigidity/strength with strategic flexibility. Further testing and optimization of this fishbone design is warranted.

### Study Objectives

In a majority of patients with total hip replacements, adverse stress conditions result in early loosening of the femoral component. Debonding/detachment observed as proximal-lateral radiolucency between the cement/bone and the prosthesis, is the first step in the loosening process, followed by extensive osteolysis [1]. These unsuccessful outcomes were attributed to both the geometry and the surface finish of the femoral component.

The scope of this study is to investigate how a debulking effort in the design of the femoral stem can act to redistribute stress patterns at the interface between the femoral component and bone. The concept is to produce a femoral component that will behave in a more similar fashion to natural femur itself, regarding its flexibility and natural stress distribution, with particular emphasis on preventing stress shielding.

In this study, the proximal half of the femurs of an older man are taken from CT scan data and converted to a 3D model in 3DSlicer. In Abaqus 2022 HF3 finite element analysis (FEA) program, this 3D model is converted to an FEA model. Using Bonemat software, the bone density and modulus of elasticity are mapped from the femur CT scan data to the FEA model. Simulations are performed under prescribed loads from ISO-7206-4:2010 starting with the natural femur, continuing with a typical solid femoral implant, and then progressive iterations of debulked femoral implants.

The goal of the FEA simulations is to produce a debulked femoral implant design that will produce general stress distribution patterns more closely resembling that of the natural femur compared to that for the solid femoral implant. At the same time, efforts are made to minimize tensile stresses to promote a successful endurance life for the debulked implant.

### Evaluation Metrics

In general, resultant stresses for both the femoral implant and the femur are within the linear elastic range for both bone and the femoral implant's material (Ti-6Al-4V); therefore, stress and strain are linearly related, and thus, both could be equally presented. In this study, stress patterns are presented in both von Mises Stress Intensity and Maximum (Tensile) Principal Stress.

The benefit of examining von Mises stress intensity is that it looks at the 3D stress “intensity” state, regardless of being compressive or tensile stresses. This is important when evaluating stress shielding, because naturally some portions of the femur will be under tensile and some under compressive stresses. Just as important is looking at the maximum (tensile) principal stress. Plotting this stress will show only the highest tensile stresses, which is what drives fatigue failures. Whereas a tensile stress will act to open the crack faces and grow the crack under sufficient load, compressive stresses will force two mating crack faces to close and, resultantly, retard further crack growth.

Model loads are applied in accordance with the ISO 7206 Standards ([2], [3], and [4]), with ISO 7206-4:2010 being most applicable for fatigue conditions and ISO 7206-10:2018 for static load rating.

### Modeling Obstacles

The proximal portions of both femurs are provided in an inherently pre-meshed geometry of tetrahedral elements. The provided femur CAD geometry has rough tetrahedral shapes, a likely result of export in .STL format, and subsequent conversion to .STP. Additionally, the femur contained numerous “stringer” voids internal to the femurs, which prohibited the FEA mesher from creating a valid mesh. These voids were manually removed using an additional program, Star CCM+, but the jagged surfaces remained.

The desire for this project was to ream the cavity of the femur and then force the implant into this reamed space to produce an interference/frictional fit, as routinely performed in these type surgeries. However, the jagged surface elements of the Zimmer implant geometry created in SolidWorks became quickly distorted and failed to converge in the solution. In lieu of the interference fit, the bone was bonded to the implant. Further studies added a cement bonding, where a layer of cement, a couple to few millimeters in width, was then created and placed between the bone, which was reamed even wider, and the implant outer surface.

An additional downfall of the rough original CAD implant geometry is that this created many high-aspect ratio elements. These “warning” elements quickly concentrate false stresses, presenting much higher stresses than what truly exists. Thus, it is imperative that when observing the stress plots, that general areas of stress be compared, and not simply look at the single highest values of stress. There are some cases with “cleaner” meshed geometries, that the peak stress of maximum principal stress is identified and referenced.

Once the cement layer was created, it was observed that this produced a much smoother stress profile than the original femur surface. Thus, for most final studies, when evaluating the implant directly in the bone, with no cement layer, the properties for this intermediate layer were changed to that for bone. However, as Bonemat divided the mass/elasticity properties into over 300 material layers, the ability to match well the femur bone at the reamed center to that of this cement layer was impossible. For sake of simplicity, the bone properties for the intermediate layer were treated as the medium layer values.

With the per-meshed state of the provided proximal femurs, a large number of tetrahedral elements were required (in the general vicinity of 1-million elements). Thus, the use of the higher quality quadratic elements was not feasible. Linear tetrahedral elements were used for the femur. Typically, this is not desired; however, with the high element count, the loss of accuracy is minimized. Visually though, the linear element stress output is less appealing. The engineer on record for the work herein has performed numerous years of studies of on FEA testing of spinal implants. In some cases, with a large degree of contact, the use of linear tetrahedral elements is a necessity. These spinal FEA tests are routinely followed up by physical laboratory testing. With suitable element density, the results of the linear tetrahedral elements were found acceptable. It should be understood that this is a comparative analysis, where the femur is meshed once and then re-used in all subsequent analyses. Therefore, even if the stress results are not truly accurate, they will provide reliable results on a comparative basis. As the lower portion of the femurs were not provided, it was modeled in 3D CAD as best as practical. There is some mismatch between the lower and upper femurs. Tie constraints are used and this type of constraints can generate high false stresses when such partial mismatches occur.

During the iterative stage of the fishbone design, the shell of the implant was retained in the model, with the internal skeletal components swapped in and out. The components of the internal framework are joined to the implant outer casing via tie constraints. The curvatures of the casing and skeletal components have difficulty aligning properly, especially at model curvatures, which creates some high false stresses due to the mismatches. Upon the final design evaluation, the implant is made solid, and tie-constraints are no longer required. This then removes the high false stresses.

It is important to understand that when tie-constraints are used and/or a component is evaluated without fillets (radiuses), then these are not accurate peak stresses. Fatigue analysis requires the accurate establishment of peak stresses. This type of stress is mesh and geometry sensitive, as well as sensitive to the surface finish of the component, manufacturing factors, material homogeneity, and other factors.

Additive manufacturing and castings will produce voids in the material, which will inherently make it weaker in fatigue than traditional fabrication methods made from solid plate or bars. A corner (90°) feature in a component will create a point of singularity, which cannot capture the true peak stress. A small fillet (radius) must be added in order to properly capture this peak stress.

Static load ratings can be established with the use of tie-constraints and without the use of the small fillets. Actually, the omittance of the small fillets is desired for static ratings test because this allows for a more homogenous overall mesh.

### Femoral Implant Related Damages

Aside from the loosening of the femoral components, consideration must also be given to other typical periprosthetic failures associated with total hip arthroplasty. FIG. 1 [2] details the more common fractures. Stress patterns in this current study take into consideration these different modes of failures.

### The Femur and its Material Property Derivation

CT scan data from the proximal half of both femurs from an older male were converted to 3D CAD format. Internal stringer voids in the CT Scan data were removed using CAD editing features in the Start CCM+ program in order to make the models useful in the FEA program. With the FEA model created for both left and right femur segments, both the CT scan data and the FEA models with the femurs were imported into Bonemat. Within this program both variable density (p) and variable modulus of elasticity (E) are mapped from the CT scan data to the femurs. Three p-E relationships are evaluated:

$$Empirical equation E = 14664 {RhoAsh}^{1.49} ( Morgan et al [ 7 ] ) \qquad 1 )$$

$$Elasticity equation for human femoral neck E = 6850 {RhoAsh}^{1.49} ( Morgan et al [ 7 ] ) \qquad 2 )$$

$$Elasticity equation derived in Javid Dissertation E = 11644 {RhoAsh}^{1.31} ( Samad , Javid , North Dakota State University [ 8 ] ) \qquad 3 )$$

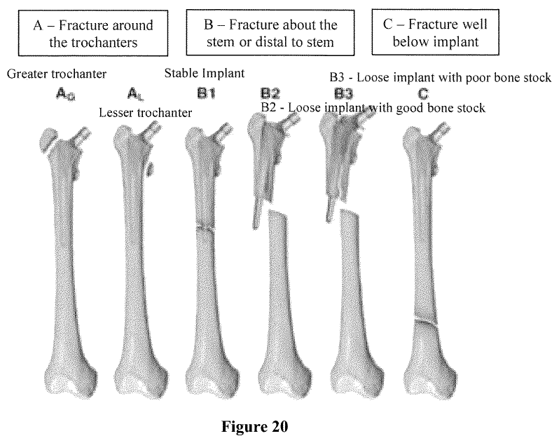

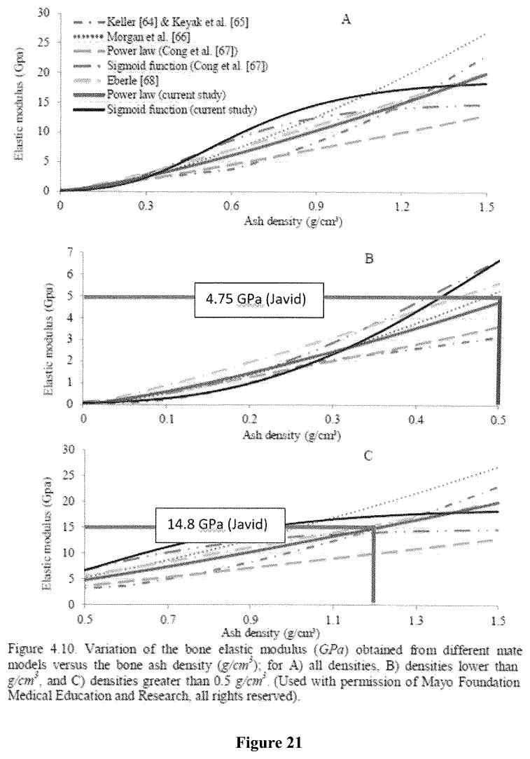

FIG. 20 the Vancouver classification of periprosthetic fractures of the femur about a total hip arthroplasty (Image from [5])

Morgan equation (1) was identified as excessively flexible and was discarded for the work herein. Both Equations (2) and (3) produced similar results. Bone mass/density for humans cover a wide range of values, which makes it difficult to discern which equation between (2) and (3) would be more applicable. By being able to use the validation efforts in the preceding paragraph, the use of Javid's equation (3) is chosen.

Within Javid's dissertation [8], seven E-ρ equations are plotted. Two representative density values (0.5 and 1.2 g/cm³) were taken from the Bonemat exported data file (.inp Abaqus format). The corresponding E values with these densities are plotted against the curves of these seven equations. [n both cases, the Javid equation falls in the mid-range of the curves. FIG. 2 shows this set of plots. The two selected E-p sets of data are just two of the over 300 material data regions outputted by Bonemat for Javid. They do not provide any net or averaged sense of the selected femur.

The output for density from Bonemat is in units of g/cm³. For this data to be accurate in the FEA SI-mm model, these units must be converted to tonne/mm3 per the following equation line:

$$1 ( \text{g/} {cm}^{3} ) {( 1 cm / 10 mm )}^{3} ( 1 E - 6 tonne \text{/lg} ) = 1 E - 9 tonne / {mm}^{3}$$

The distal femur segment is 3D CAD model using a representative femur outline. There is no CT scan data for this femur extension. Thus, it is assigned the generic bone properties shown in Table 1. In the implant-to-bone studies to follow, the cement region, with its higher quality elements, was changed to bone properties. The medium Bonemat property layer (Mat-155) is used (also shown in Table 1). Values used for the femur were established early on based on numerous published data.

TABLE 1
Generic bone properties for distal femur extension segment

| Property | Femur | Medium Bonemat |
|---|---|---|
| Density | 0.31 g/cm3 | 0.764 g/cm3 |
| Modulus of Elasticity | 14700-MPa | 8237-MPa |
| Poisson's Ratio | 0.3 | 0.3 |

FIG. 21 shows two Javid E-p Results Compared against Seven E-p Equations (FIG. 4.10 of [8])

### Femoral Implant Material Properties

Titanium Grade 5 (Ti-6A1-4V) is used for all femoral implant components.

TABLE 2
Ti-6A1-4V (Grade 5) Material Properties

| Property | Value |
|---|---|
| Density | 4.43 g/cm3 |
| Modulus of Elasticity | 113,800-MPa |
| Poisson's Ratio | 0.342 |
| Yield Strength | 880-MPa |
| Ultimate Tensile Strength | 1100-MPa at plastic strain of 0.014 |

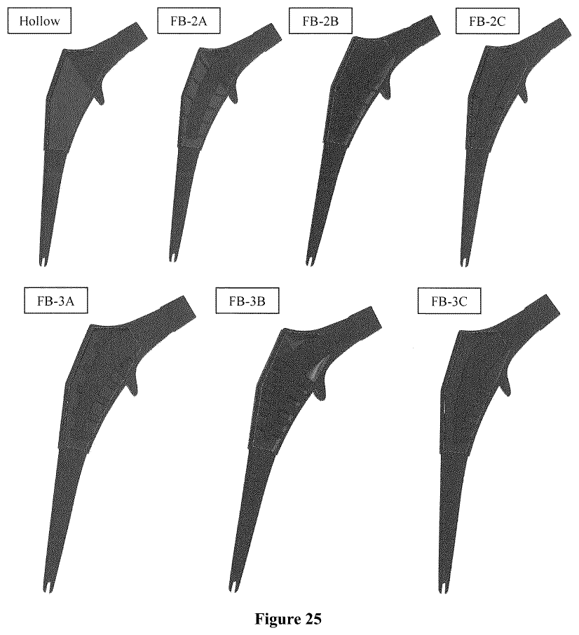

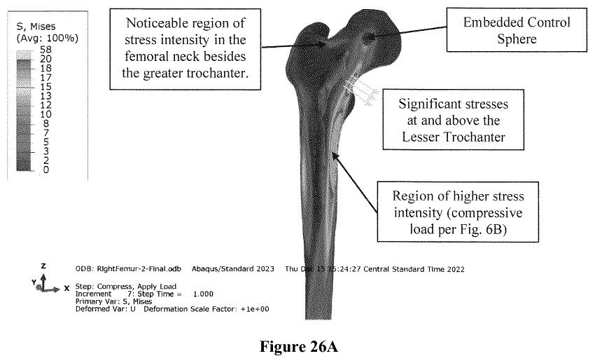

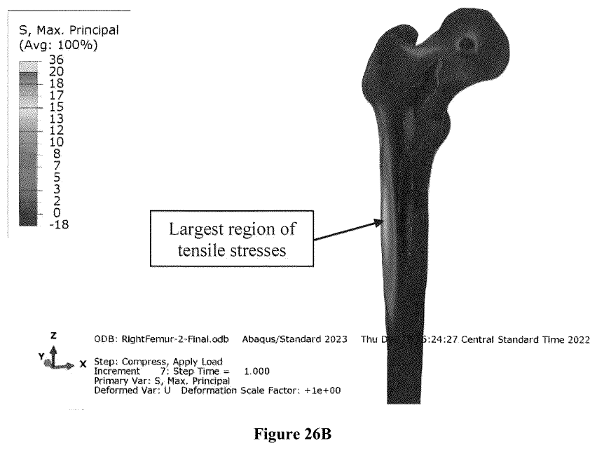

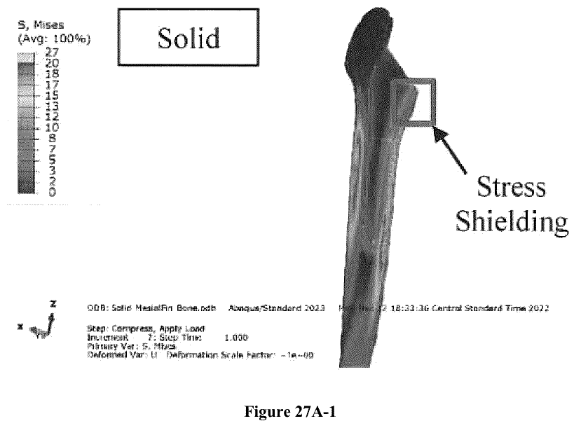

Ti-6A1-4V is evaluated for fatigue failure using “fatigue curves”, which are specific for material, fabrication processes, grain-orientation, testing medium, textures, etc. For Ti-6Al-4V, with the worst published texture curve in air is used. It is taken from ASM Handbook “Volume 19: Fatigue and Fracture” [9], p. 842, FIG. 25 (shown in this report as FIG. 3). This fatigue curve shows that at 10 million cycle run-out, an alternating amplitude stress of 600-MPa is required. A Keq is used for Ti-6A1-4V to account for saline environment testing, manufacturing technique and quality, and other potential surface irregularities. A single “Keq” factor is created from the various contributing “k” factors and is derived from numerous comparisons of the FEA results to laboratory test results for particular products and manufacturing processes, and vendors.

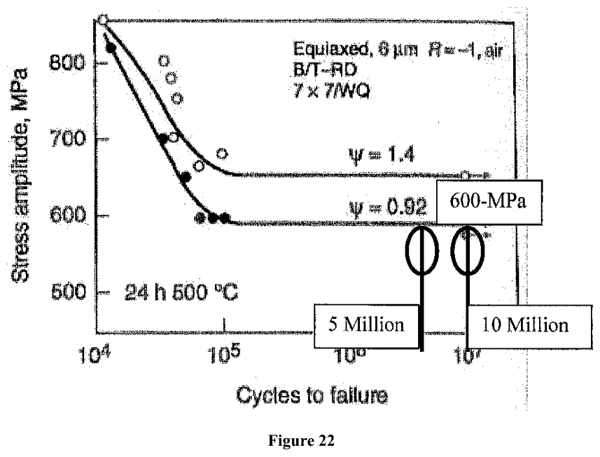

FIG. 22 shows S-N curves in Tl-6Al-4V. Effect of degree of texture.

### FEA Model Setup

The left femur is cut and reamed to allow for insertion of the femoral implant, along with cement. Also included in the model is the femoral head and the acetabular cap (but not the liner). Only the proximal region of the left femur was available from CT scan data. The distal region of the left femur was then completed in 3D CAD using visual references from a full human femur (NIH 30 Print Exchange). FIG. 4 (left) provides an overview of the FEA meshed model. A cross-sectional cut-away in FIG. 4 (right) shows the layout of the femoral stem and the applied cement around the femoral stem.

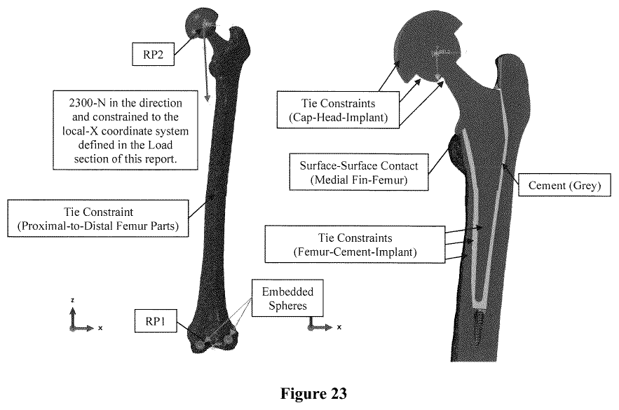

FIG. 23 shows FEA Model Mesh and Materials (Left: Full 3D Model; Right: Cross-Section View)

### Meshing, Material Models, Part Tie-Constraints, and Part-to-Part Contact

All devices use quadratic tetrahedral meshing, except for the acetabular cap which is swept linear hexahedral meshed. The femur and the femoral head are meshed with a 1-mm mesh size, while the femoral body and cement use a 0.75-mm mesh size.

Material properties (modulus of elasticity (E) and density (p)) for the femur are derived from CT scan data and mapped using Bonemat software. Poisson ratio (v) is 0.3. All metal components are Ti-6Al-4 V. A linear strain hardening plasticity is applied to this titanium with the yield point set to 880-MPa and the ultimate true stress set to 100-MPa at an ultimate true strain of 0.014.

As shown in FIG. 4, part tie-constraints are used to bond parts together, while surface-to-surface contact is used between the implant medial collar and the mating femur surface.

### Boundary Conditions, Couplings and Spatial Model Constraints

Two kinematic couplings are used in this FEA model. The first has the condyles (modeled with two embedded spheres) of the distal femur with the two spheres controlled by a reference point centered between the lateral and medial condyle. The second coupling has the outer surface of the acetabular cap controlled by a reference point located at the center of the femoral head. FIG. 4 (left) identifies these two control reference points. Loading and displacement-controlled constraint is applied to the RP2, while RP1 is fixed in space. For RP2, both the loading and displacement-controlled constraint are assigned to a local coordinate section, derived in the next section. For the displacement-controlled constraint, RP2 is only allowed to move along the X-axis of the local coordinate system, simulating the control the hip has on the femur head. At the knee joint, RPI is fixed in space, meaning the displacement and rotation of both embedded spheres are prohibited from moving.

### Loads

In order to apply the required loads per ISO 7206-4:20 10, both existing alpha, the angle in the frontal plane CKL (FIG. 5) between the load axis and the stem axis, and beta, the angle in the lateral plane perpendicular to CKL between the load axis and the stem axis, must be determined. FIG. 5 uses the 3D CAD model to sketch both the planes and identifies various axes used to make the angle determinations.

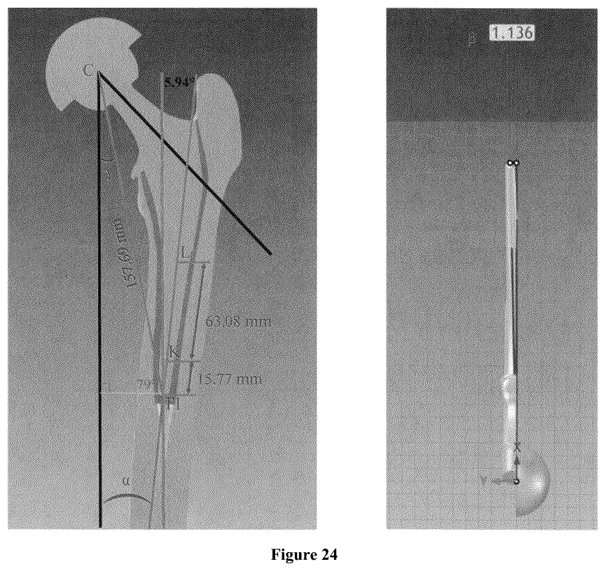

FIG. 24 shows Measurements for Alpha and Beta per ISO 7206-4:2010 (Figure A.3) From the 3D CAD model, the following data points were taken:

### Point C (56.426, 28.333,−385.637)

### Point Tl (82.198, 43.858, −540.428)

Length CT between the two points is the following:

$${L}_{CT} = \sqrt{{( 56.426 - 82.198 )}^{2} + {( 28.333 - 43.858 )}^{2} + {( - 385.637 + 540.428 )}^{2}} = 157.9 mm$$

The angle from vertical of the two points is the following:

$$N = {z}_{1} - {z}_{2} = - 385.637 + 540.428 = 1 54.79 mm$$

Using direction cosines, γ=arccos (N/L_{CT})=arccos (154.79/157.69)=11.0-deg
Angle between Line TKL and the vertical is measured in 3D CAD to be 5.94-deg. Therefore, using trigonometric relationship, α=5.94-deg as well.

Due to the irregular spatial positioning of the femurs originally established in the CT Scan data, beta is measured directly in 3D CAD program. Beta, 1.14-deg, is the angle in the lateral plane perpendicular to CKL between the load axis and the stem.

### Creation of Load Direction Local Coordinate System

In order to produce loads at α=10° and β=9° per Table 1 of ISO 7206-4:2010, a must be increased by 10-5.94=4.06° and β must be increased by 9-1.14=7.86°. Using the “C” coordinate (56.426, 28.333, −385.637) as the origin of the new local Cartesian coordinate system, and maintaining L_{CT}=157.69 mm, the new T2 coordinate is established by rotating both a and B by the prescribed amounts to achieve ISO 7206-4:2010 requirements. The resulting T2 coordinate is the following:

T2(62.932,54.124,−541.065)

Hence from C (56.426, 28.333, −385.637) to T2 (62.932, 54.124, −541.065) establishes the X-axis in the new local coordinate system. The ISO 7206-4:2010 load of 2300-N for 120<CT≤250 is applied in the local coordinate system along this local X-axis. Both Y- and Z-axes are irrelevant for this single direction loading. The use of T2 coordinate does not imply that the stem is moved. It simply provides the necessary line of action to apply the load in a derived local coordinate system.

### Solid, Hollow and Fishbone (FB) Implant Model Descriptions

Implant models started with the solid version, followed by the creation of a hollow implant with wall thickness of 2-mm. The Version 1.0 fishbone was an as-received version that was not conducive to optimization and testing. A new fishbone structure was created in Version 2.0 (A-C); this version contained thin, curved trusses emanating from the fishbone spine. With Version 3.0 (A-C), the trusses were switched to flat projections and were significantly thickened, some with variable thickness. FIG. 6 shows a cross-section view of each of the models included, minus the solid implant, in the testing herein.

FIG. 25 shows hollow and Fishbone Cross-Sectional Views.

### Natural Femur Stresses Under ISO 7206-4:2010 Loads

A load of 2300-N is applied as shown in FIG. 4, following the X-direction of the local coordinate system defined from ISO 7206-4:2010. Loading the natural femur was accomplished by embedding one small sphere, centered in the femur head. The load and directional constraint are applied to a reference point at the center of this sphere, controlling its motion. FIGS. 7A/B show, respectively, the von Mises stress intensity and the maximum (tensile) principal stress plots. All femur stress plots are set between 0 and 20-MPa for comparative purposes. Evaluation of stresses should be on general distribution, and not simply looking at the maximum values (which could be false readings).

FIG. 26A shows a natural Femur-von Mises Stress Intensity Plot.

FIG. 26B shows a natural Femur-Maximum (Tensile) Principal Stress Plot Implanted Femur Stress Under ISO 7206-4:2010 Loads

Repeating the FEA simulations with identical setup and loads is now conducted for the solid, hollow, and fishbone implants. Two sets of simulations are conducted for each. The first considers implant-to-bone bonding (FIG. 8A/B), while the second set considers the use of cement between the reamed bone canal and the implant outer surface (FIG. 9A/B). For each set of simulations, plots are created for the von Mises stress intensities (“A” images) and maximum principal (tensile) stresses (“B” images).

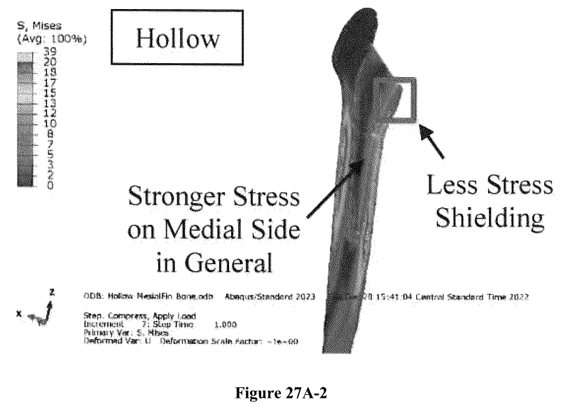

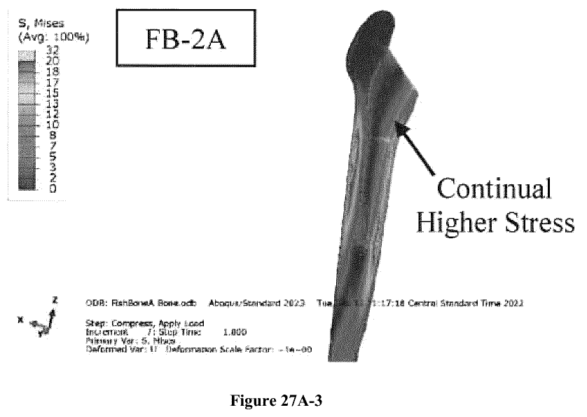

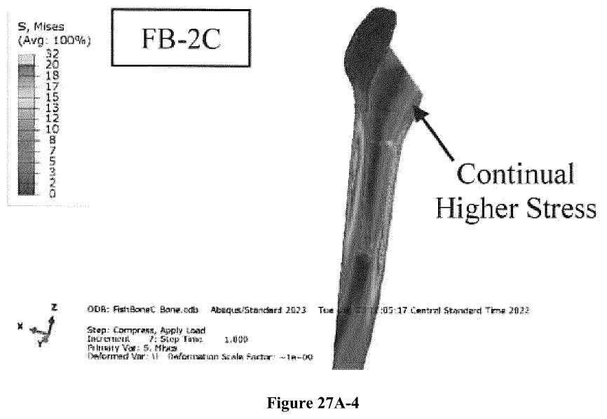

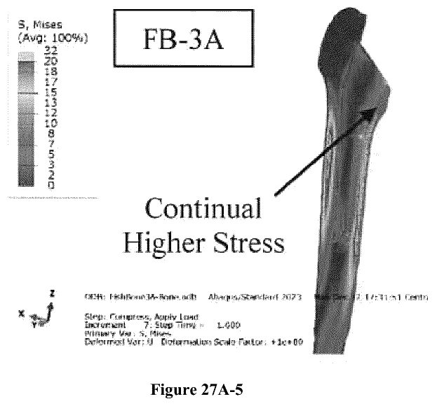

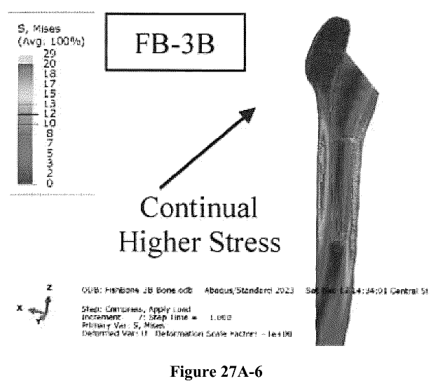

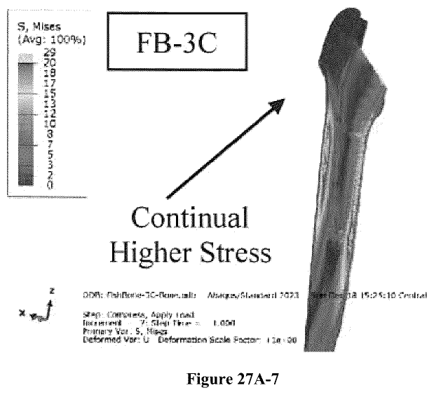

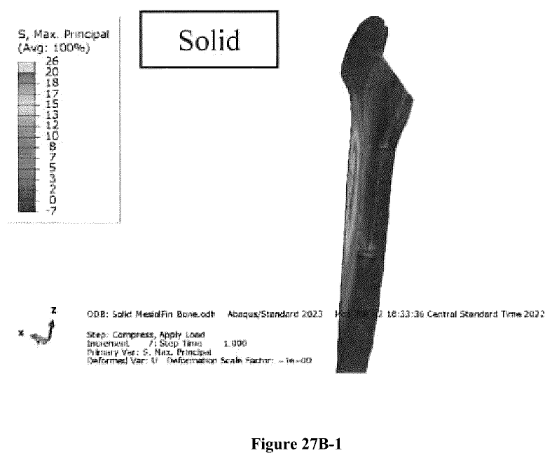

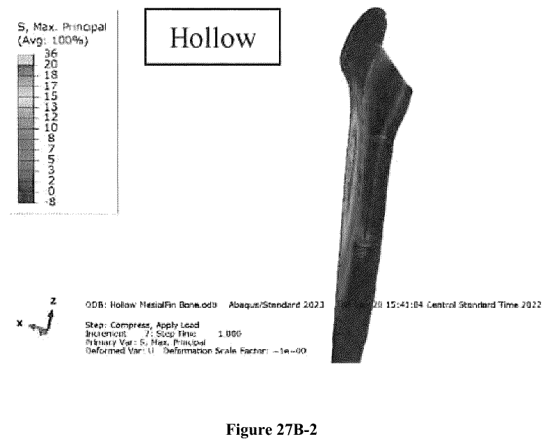

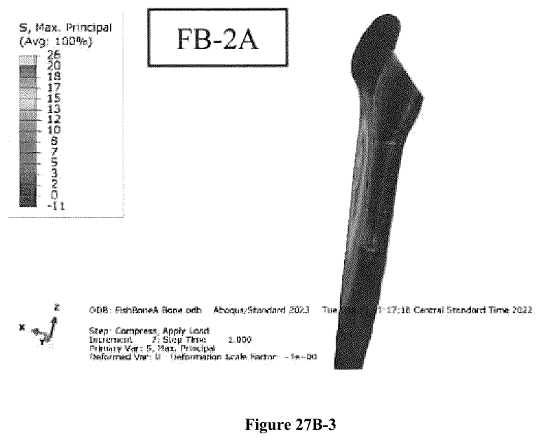

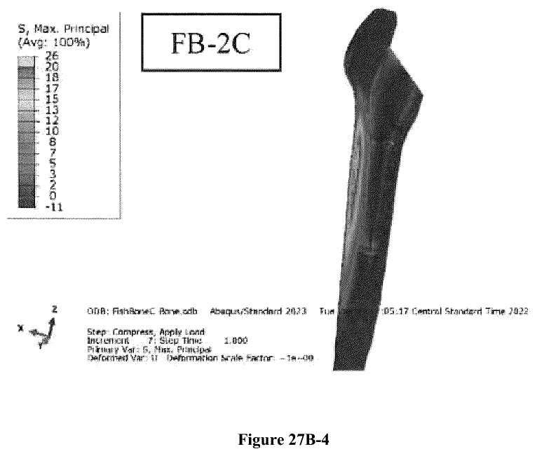

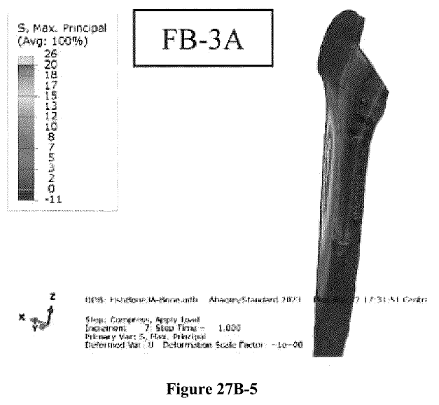

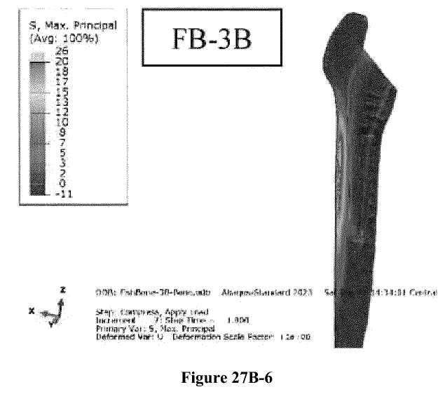

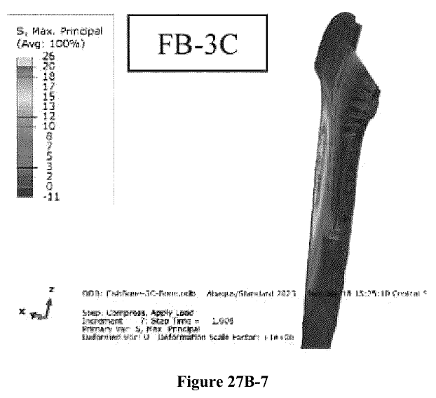

FIGS. 27A-1-27A-7 shows Von Mises Stress Intensity in Implanted Femur with Bone/Ti Contact.

The benefit of any degree of implant debulking is seen in FIG. 8A. In the boxed area for the solid implant, the almost negligible stress level “blue” is seen. With the debulking in the remaining images, a higher general “green” stress is observed, with the Hollow design having the least stress shielding. The FB3A-C designs have more noticeable stress on the anterior/posterior sides of the femur due to the larger trusses in these designs.

FIG. 27B-1-27B-7 shows maximum Principal (Tensile) Stresses in Implanted Femur with Bone/Ti Contact.

All images for maximum principal stresses are quite similar, and 1a1 designs shoed the maximum. In this set of images, looking at the maximum stress is suitable. Notice that except for the Hollow design, all designs have the same maximum stress, 26-MPa. However, a comparison of stresses in bone at the proximal-lateral junction suggests a nominal, but observable, increase in tensile stresses besides hollow and various truss designs compared to the solid design.

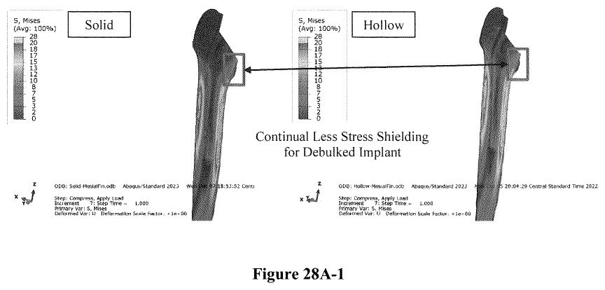

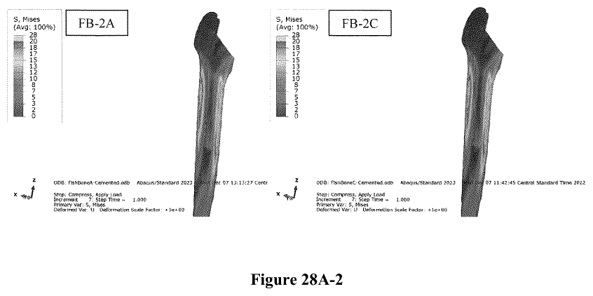

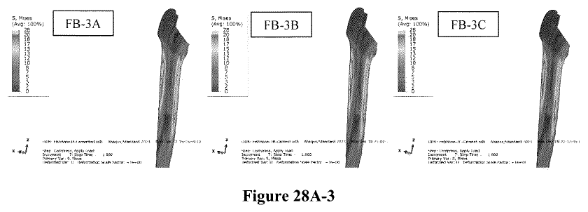

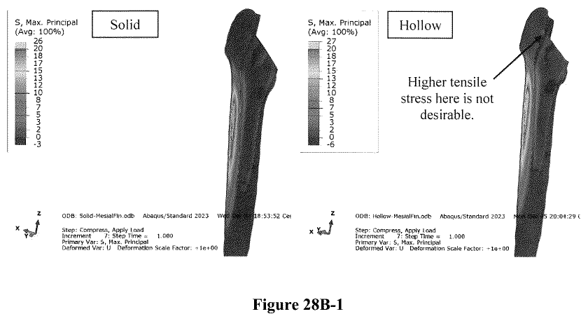

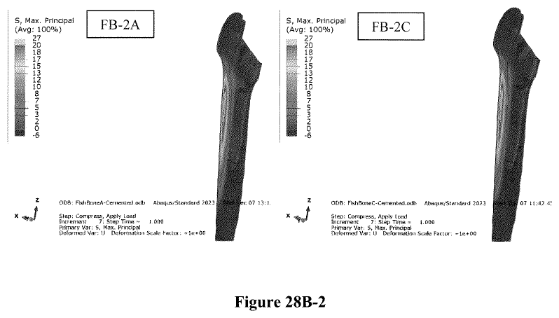

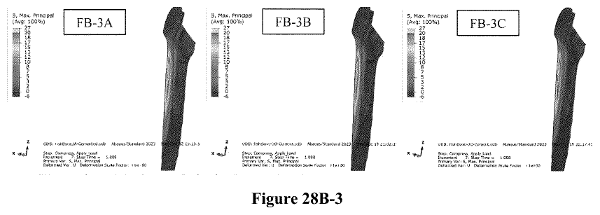

FIG. 28A-1-28A-3 shows Von Mises Stress Intensity in Implanted Femur-Cemented.

The cement is flexible with a low modulus of elasticity. It acts to buffer and redistribute the implant loading to the femur. However, there is still a noticeable reduction of stress shielding near the medial-proximal region with tested designs compared to the solid configuration.

FIG. 28B-1-28B-3 shows maximum Principal (Tensile) Stresses in Implanted Femur—Cemented.

Again, the stress profiles are strikingly similar with the exception to the localized higher tensile stress at the proximal-lateral connection between the cement and the femur. These stresses act to de-bond the cement from the femur. The hollow design is the poorest of the designs. To a lesser degree, the fishbone designs also show incrementally higher stress in this region compared to the solid implant.

### Cement Debonding

As pointed out previously, one potential downfall of the fishbone design is the higher tensile debonding stresses developed in the proximal-lateral bond joint. Using tests data from (Table 1 of [6]), the mean tensile strength for bone-cement bonding is 6.3-MPa, with a minimum test value of 2.4-MPa and a maximum of 10.2-MPa. The bonding between Ti-6Al-4V and cement was significantly higher, with a mean tensile strength of 14.9-MPa (8.2-MPa min). Using the results from the previous models, the cement-bone interface (bonding surface in the FEA model), is isolated and maximum principal stress is plotted on a scale of 0-2.4 MPa, the lower bound cement-bone tensile strength. FIG. 10 provides a comparison of these maximum tensile principal stresses. The common theme is that the stiffer the implant, the lower the cement-bone maximum tensile strength: 4.1-MPa for the solid, 4.4-MPa for the stoutest fishbone (FB-38), to 4.5-MPa for the rest of the fishbone implants, to 5.3-MPa for the hollow design. All implants have significant tensile stresses above the minimum debonding strength of 2.4-MPa.

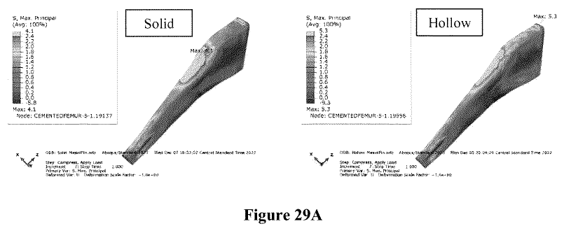

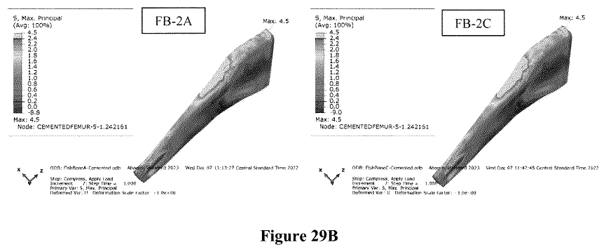

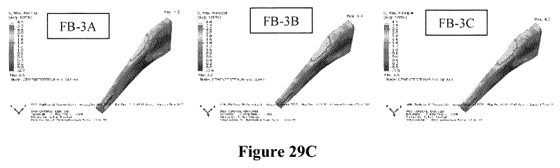

FIG. 29A-29C shows maximum Principal Stresses at Cement-Bone Interface Implant Maximum (Tensile) Principal Stresses.

A screening for fatigue of the implant designs is made by plotting maximum principal stresses of just the implant. FIGS. 11 and 12 show, respectively, the stress results for the cemented and the bone/implant designs. The two most flexible design (hollow and FB-2C) had the lowest peak stresses, while the most rigid solid design and other designs with highly constrained fishbone trusses had the highest peak stresses. FB-3C had the best compromise of rigidity and select flexibility.

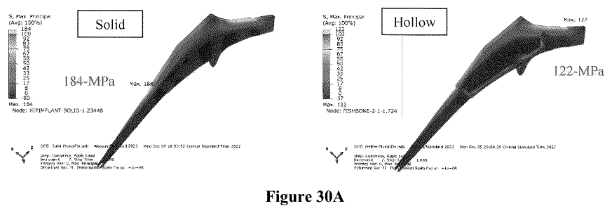

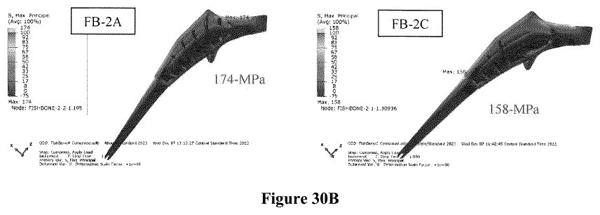

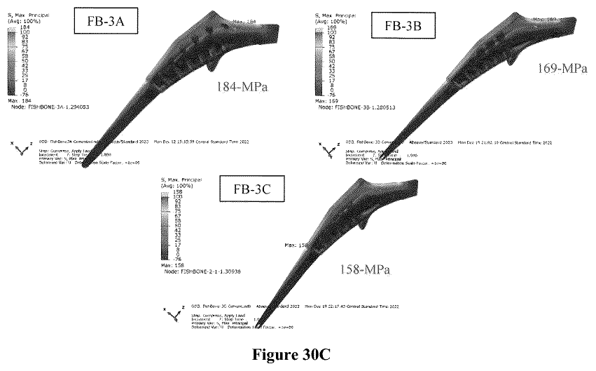

FIG. 30A-30C shows Maximum Principal Stresses in Ti-6Al-4V Implant when Cemented.

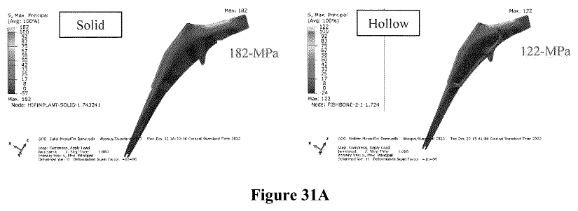

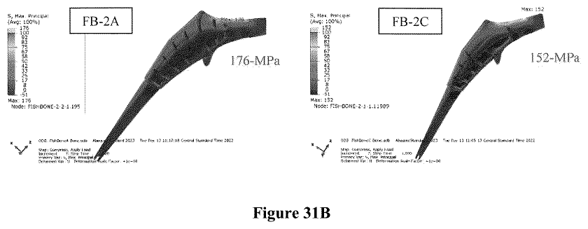

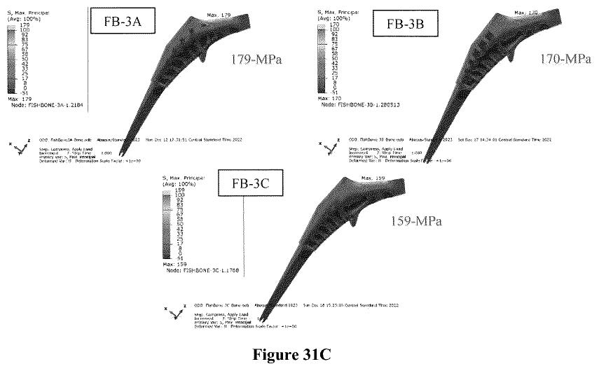

FIG. 31A-31C shows Maximum Principal Stresses in Ti-6Al-4V Implant when in Contact with Bone.

Once again, but now even more pronounced, the rigid solid implant has much higher peak stress (182-MPa) compared to the most flexible hollow implant (122-MPa). FB-3C remained the best balance of rigidity and selective flexibility with a peak stress of 159-MPa.

### Medial Collar vs No Medial Collar

In all preliminary studies, the medial collar was included with the implant. The following images in FIGS. 13A/B and 14A/B provide a brief evaluation of the effects of removing this collar. FIGS. 13A/B show the results for the cemented models and FIGS. 14A/B show the results for the bone-Ti models. “A” images are von Mises stress intensities and “B” images are maximum principal stresses. Cement bonding is revisited in FIG. 15, and maximum principal tensile stresses for select implants shown in FIG. 16 when medial collar is removed.

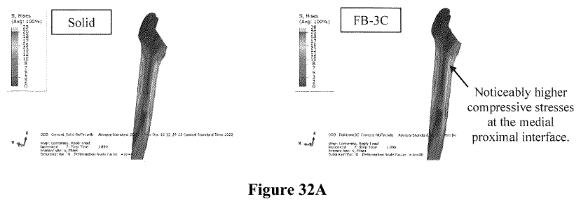

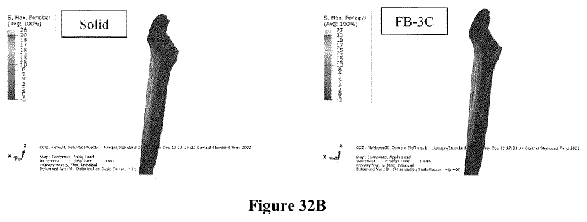

FIG. 32A shows No Medial Collar—Cemented—von Mises Stress Intensities.

FIG. 32B shows No Medial Collar—Cemented—Maximum Principal Stresses.

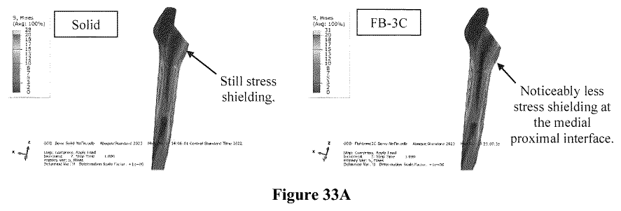

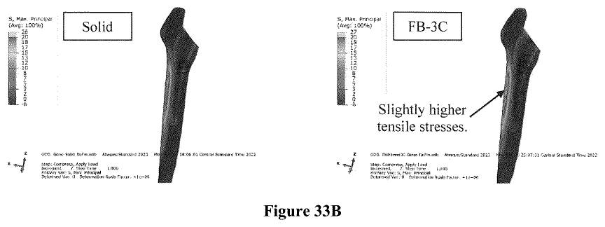

FIG. 33A shows No Medial Collar—Bone/Ti—von Mises Stress Intensities.

FIG. 33B shows No Medial Collar—Bone/Ti—Maximum Principal Stresses.

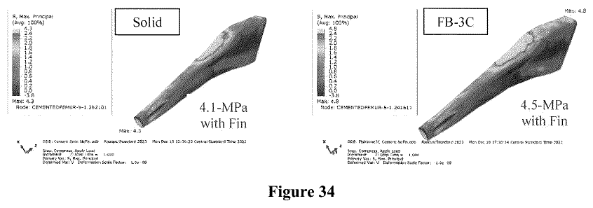

FIG. 34 shows No Medial Collar—Cement—Bone Bond—Maximum Principal Stresses.

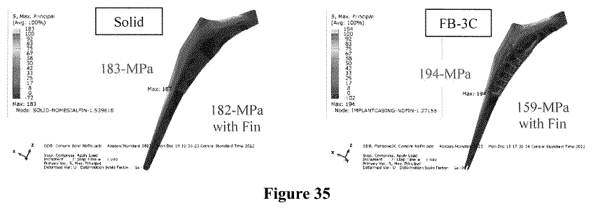

FIG. 35 shows No Medial Collar—Implant—Maximum Principal Stresses.

The solid implant is only slightly changed when the medial collar is removed, with the mostly noticeable change being the increase in cement-bone bonding stress from 4.1-MPa to 4.3-MPa. On the other hand, the FB-3C appears poorer without the medial collar. Most noticeable is the increase in implant stress from 159-MPa to 194-MPa, as well as the increase in cement-bone bonding stress from 4.5-MPa to 4.8-MPa. Also, both von Mises and maximum principal stresses show increases in stress levels. Although higher stresses are desired in areas of stress shielding, increasing the bonding stress and increasing the peak implant stress are not desirable. The fishbone models should be designed with the medial collar.

### Implant Static Load Tests (ISO 7206-10:2018)

The solid implant and both FB-38 and FB-3C implants are now removed from the femur and potted in a test cylinder. Delrin (density=1.41 g/cm³, E=3100-MPa, and v=0.35) is used as the potting material. This is a simple test where the implant, potted in the test cylinder, is loaded in the same manner as the previous 7206-4:2010 tests, but now loaded in displacement-controlled manner until failure. That is, the acetabular cap is forced to follow the X-direction in the local coordinate system for an arbitrarily chosen 50-mm. A load-vs-displacement chart is created, where the failure load, in this case, is easily observed.

In each image of FIGS. 17-19, the top image is point of loading which is best visually for understanding of the failure mode. Notice that the legend is set to 0.014 (or 1.4%) equivalent plastic strain (PEEQ). This is the ultimate failure strain. Notice that the image chosen for plotting has PEEQ in the hundreds of percent, indicating that failure had already occurred in this relatively brittle Ti-6Al-4V material.

All devices are suitably strong based on this plastic-based analysis, with the solid implant failing at 85-kN due to neck gross yielding, while both fishbone implants failed at 63-kN due to local outer casing buckling. The previous ISO 7206-4:2010 loads applied were 2.3-kN. These static values are useful for a comparative analysis, however, to ascertain the true failure strength of each, a fracture mechanics evaluation would be required.

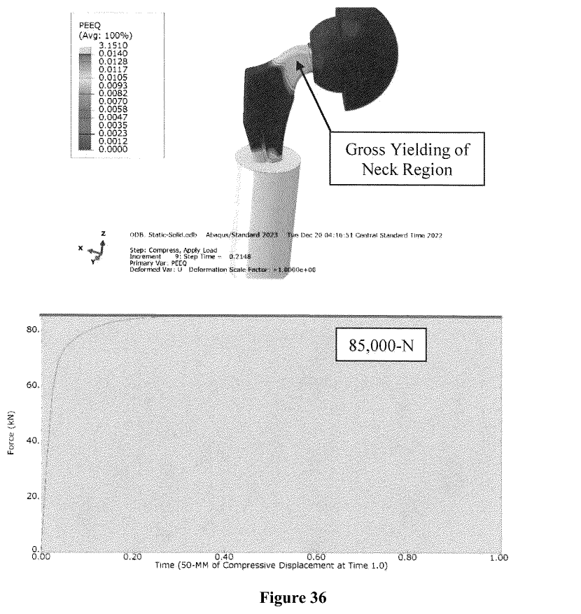

FIG. 36 shows Static Load Tests-Solid Implant LOO.

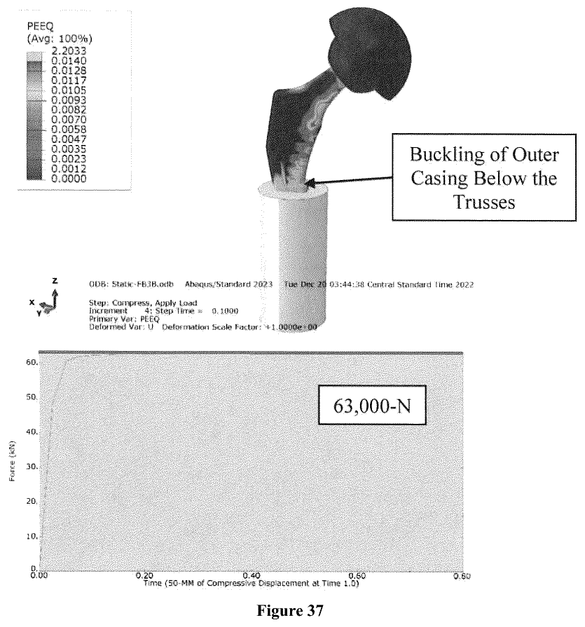

FIG. 37 shows Static Load Tests-FB-3B Implant.

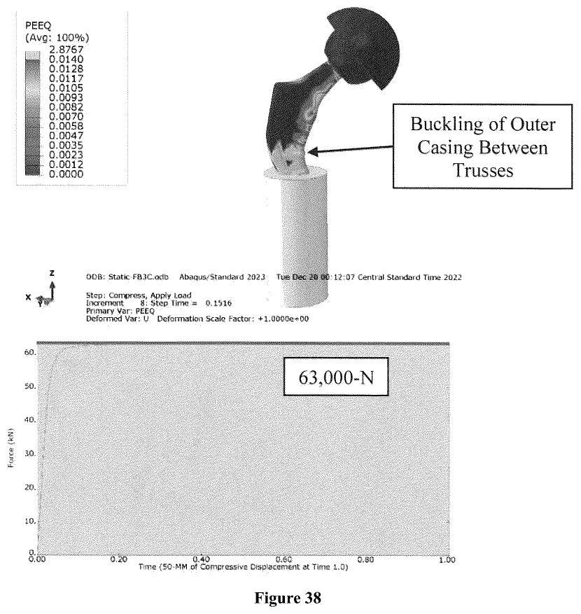

FIG. 38 shows Static Load Tests-FB-3C Implant.

### Implant Endurance Load Tests (ISO 7206-4:2010)

FB-3C now has its outer casing, central spine and trusses solidly joined together in 3D CAD, with fillets added through the inner implant cavity. The potted test cylinder for the solid test is repeated here, but now instead of applying an arbitrarily chosen displacement of 50-mm, a (cyclic) load of 2300-N is applied, being forced to follow the X-direction of the local coordinate system. Both the solid and FB-3C are evaluated for fatigue (endurance) under these loads and conditions. For these fatigue models, the stresses are mesh sensitive; however, based on years of FEA testing and comparison to laboratory testing, a mesh size of 0.05 to 0.10 mm in critical locations has been shown to provide acceptable peak stress results.

The solid implant has a peak stress located at the neck transition, with a value of 65-MPa (FIG. 20). As this is single direction loading (not reverse bending), the alternating stress amplitude is half of this value or 33-MPa. Based on numerous comparisons for Ti-6Al-4V spinal implant FEA tests to laboratory tests, a fairly high-end single K-factor is 3.0. With the 10-million endurance alternating stress amplitude equal to 600-MPa (referring to FIG. 3), the stress of 33*3, or about 100-MPa is well under the limit of 600-MPa for achieving 10 million cycles.

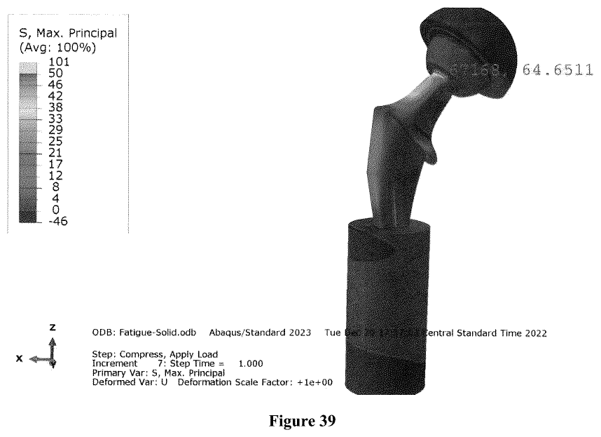

FIG. 39 shows endurance Tests-Solid Implant-Maximum Principal Stresses

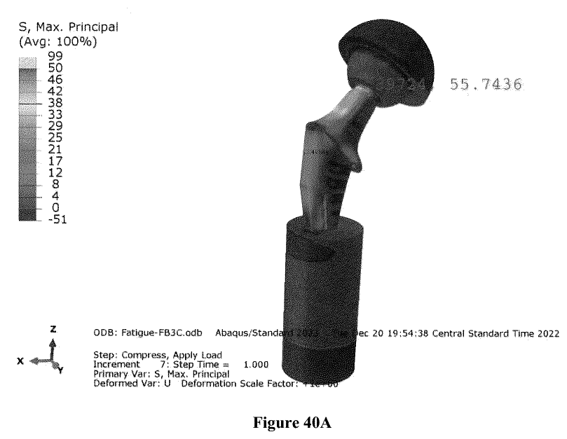

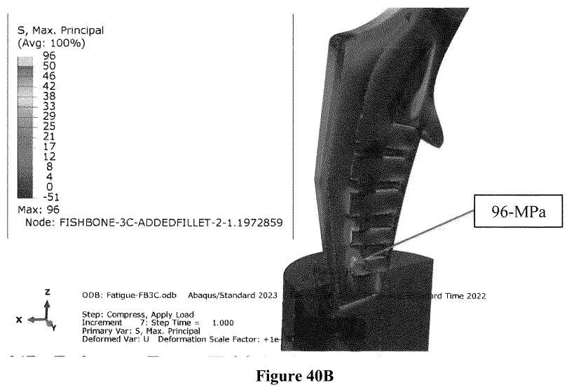

FIG. 40A shows Endurance Tests-FB3C Implant-Maximum Principal Stresses

FIG. 40B shows Endurance Tests-FB3C Implant-Maximum Principal Stresses

Due to the increased flexibility, the neck transition has a lower peak stress for the FB-3C implant (FIG. 21A). However, the lower truss of the fishbone skeleton has a higher peak stress of 96-MPa (FIG. 21B). This stress was not picked up in the femur tests. The loading is simply quite different. Whereas, in the femur, much of the load is taken by the proximal half of the implant. In this ISO test, the loading is strongly focused to the lower distal portion of the implant. Looking at the endurance calculation, but this time using a single K factor of 5.0 to account for unknowns in fabrication technique, the alternating stress amplitude of 96/2*5.0=240-MPa. With a 10-million cycle endurance limit of 600-MPa, this alternative stress value is deemed acceptable. Still, there are design methods that can reduce this peak stress of 96-MPa. These two lower trusses can be made 360° and they can be thickened to match the top trusses. Both actions in this distal location are expected to produce minimal increase in overall implant rigidity. Notice that the top trusses are all below 50-MPa.

### CONCLUSIONS

The use of solid implants shows the propensity for stress shielding at the proximal-medial junction of the implant to the femur. Debulking the implant, at first by completely debulking to a hollow design, shows that stresses can be returned to this location through the added load sharing of a flexible design. An additional critical benefit with a more flexible implant design is that, with interference fit designs, the added flexibility will allow for better distribution of stresses between the implant and reamed cavity. Without a perfect fit between the implant and reamed cavity, a rigid design will generate hard and light (or no) contact zones. The flexible design will better maintain a consistent contact load. Further, the more flexible design will be easier to install.

However, a hollow design is weak. The static and torsional strength, not tested herein would be a significant drop from the solid design. The high flexibility of the hollow design also leads to much greater damaging tensile stresses to cement bonding between the bone and implant, considering cemented designs.

To counter the weakness of the hollow design but attain some of its minimization effects for stress shielding, the fishbone design was modeled, and several variations tested. Initially developed were curved and thin trusses and a small diameter central spine. Then, the trusses were flattened and thickened. Issues were observed with those fishbone designs that had trusses located above the medial collar. These trusses became constricted and developed high stresses in the implant. FB-2C, FB-3B, and FB-3C focused the trusses below the medial collar. Still, FB-3B, with its thicker trusses became too rigid when the lateral side extended almost to the casing. FB-3C was then copied from FB-3B, but with the trusses mostly cut off on the lateral side. Ultimately, FB-3C provided the best combination of rigidity and selective flexibility, with the implant maximum principal stress being the lowest among the fishbone design, matching that of the very flexible, but weak FB-2C.

Two important observations are made for the fishbone designs. One, the added flexibility of the fishbone design imparts greater tensile stresses on the cement, when used. Such higher tensile stresses will lead to earlier debonding issues. The fishbone design should be designed for bone-implant interference fit surgical procedures. Second, the medial collar is necessary for the proper functioning of the fishbone design. Without the medial collar, implant stresses are notably higher. Although higher femur stresses could be desirable, the higher implant stresses are not.

Static strength inherently will be weaker for any debulking design compared to a solid design, of the same cross-sectional design. However, there appears to be suitable strength for the expected loads acting on the body. A fracture mechanics study is required to provide a true failure strength for this brittle material.

The fatigue study for ISO 7206-4:2010 shows that it does not truly replicate the type of loads the implant will see in the human body. Being potted quite low on the distal stem, this places a load on a part of the implant not expected to see much load. This is true with the fishbone design tested, FB-3C, where the highest peak stresses are located at the bottom truss. In the femur tests, these peak stresses were located near the neck and medial collar general areas. Still, at 48-MPa alternating stress amplitude, even considering a highly conservative single K factor of 5.0 (equivalent to a fine crack), the stress state is low enough to achieve the desired 10 million cycles of loading.

FB-3C is chosen as the best of the fishbone designs presented herein. However, further optimizations can be made to lower implant stresses even further.

### Continued Optimizations and Additional Studies

As the creation and morphing of the fishbone design proceeded, various testing setups were created and then upgraded with more realistic simulation conditions. An intent to test both male and female femurs was replaced with the industry standards of ISO 7206-4:2010 and 7206-10:2018. Still, it would be desirable to repeat several of the FEA tests herein with loads of various angulations, particularly those inducing torsion. A re-orientation of the implant to the x, y, z Cartesian coordinate system going forward will facilitate quick changes of angulations. Currently, the femur, and thus the implant, is on a skewed coordinate system.

Using an initial solid implant posed numerous obstacles. The 3D CAD model contained numerous geometry flaws, requiring extensive repairs in the FEA program for each iteration used. Going forward, it would be beneficial to create an implant from scratch. It is highly desirable to eliminate the numerous stress raisers that exists in this design. Some areas in the cavity of the implant were not able to have fillets (radiuses) to the newly added truss network. A clean implant design will allow for a fully filleted geometry to allow better capture of peak stresses.

The FB-3C design should be optimized to drop the peak stresses at the distal truss in the ISO endurance test. These stresses appear to be able to be designed out, with simple non-intrusive geometry changes.

A higher end fine-tuning would require making and testing prototypes to allow establishment of a fatigue K factor, taking into account the manufacturing process, repeatability, and material homogeneity. For a true static strength, a fracture mechanics study should be completed.

Not considered in this testing was the effect of the interference fit. For one, this will create a compressive stress field at the interference fit. Further, the design requires evaluation of stability of the outer casing under the press fit conditions, as well as process of forcing the implant into the reamed femur cavity. With the use of the cement as a quality layer of elements for bone in the femur canal, the surface smoothness of the femur cavity was greatly enhanced. This modification to the femur could allow for the interference fit study to take place. However, the numerous discontinuities of the current implant outer design remain as obstacles. With a redesigned outer casing of the implant, this study may be possible.

### Abbreviations

FEA—Finite element analysis
SI—International system of units (Système International d′Unités)
E—Modulus of Elasticity
v—Poissons Ratio

### Medical Terms

Periprosthetic—a structure in close relation to an implant.
Proximal and Distal—Proximal referring to closer to the torso. Distal referring to away from the torso.
Medial (Medial) and Lateral—Medial (Medial) refers to toward the center of the body and lateral away from the center of the body

### REFERENCES

1. Mohler C G: Early loosening of the femoral component at the cement-prosthesis interface after total hip replacement. J Bone Joint Surg Am. 1995 September
2. ISO-7206-4:2010: Implants for surgery—Partial and total hip joint prostheses—Part 4: Determination of endurance properties and performance of stemmed femoral components. 3^{rd} Ed., 2010.
3. ISO-7206-6:2013: Implants for surgery—Partial and total hip joint prostheses—Part 4: Endurance properties testing and performance requirements of neck region of stemmed femoral components. 2^{nd} Ed., 2013.
4. ISO-7206-10:2018: Implants for surgery—Partial and total hip joint prostheses—Part 4: Determination of resistance to static load of modular femoral heads. 2^{nd} Ed., 2018.
5. Nauth A, Stevenson I, Smith M D, Schemitsch E H: Fixation of periprosthetic fractures about/below total hip arthroplasty. Musculoskeletal Key. 2016.
6. Beckmann N A, Bitsch R G, Jaeger S: Mechanical properties of a cemented porous implant interface. Acta Orthopaedica. 2013.
7. Morgan E F, Bayraktar H H, Keaveny T M. Trabecular bone modulus-density relationships depend on anatomic site. Journal of biomechanics. 2003.
8. Javid S: Optimized evaluation of bone tissue material properties by inverse finite element analysis and femur fracture testing. A dissertation submitted to the graduate faculty of the North Dakota State University of Agriculture and Applied Science. 2014.
9. Lampman S R: ASM Handbook. Volume 19: Fatigue and Fracture. ASM Intl. 1996.

### Example 3

The embodiments described above include controlled deformation for purposes of load transfer. The loading of an implant causes an outer shell to reversibly stretch/deform/buckle resulting in the transfer of compressive and tensile stresses to bone. Under an embodiment, the concept may be applied to a dental implant.

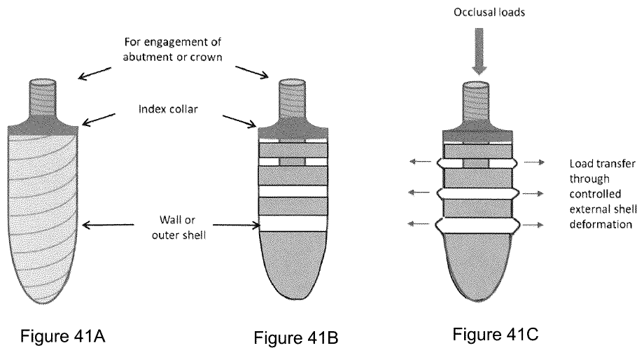

FIGS. 41(A)-(C) show schematic diagrams of the screw-based dental implant. A. External view, B. Vertical sectional view, and C. Vertical sectional view under applied occlusal load. Strategic hollowed out regions within the implant accommodate flexing of the outer shell when occlusal loads are applied. The shaft end that attaches to the abutment or crown is either threaded or has a different mode of engagement. The index collar seals the base.

1. A femoral stem device comprising,
a stem and a neck, wherein the stem comprises an outer shell, wherein the stem comprises a proximal region and a distal region, wherein an interior of the proximal region is selectively hollowed, wherein a loading of the device comprises deforming laterally disposed walls of the proximal region outer shell.

2. The femoral stem device of claim 1, wherein the deforming comprises lateral deformation of the walls adjacent to the selectively hollowed spaces.

3. The femoral stem device of claim 1, wherein the selective hollowing comprises an entire hollowing of the proximal region interior.

4. The femoral stem device of claim 1, wherein the selective hollowing comprises a partial hollowing of the proximal region interior.

5. The femoral stem device of claim 4, wherein the partial hollowing provides a curved central spine.

6. The femoral stem device of claim 5, wherein the curved central spine is aligned with a curved longitudinal axis of the stem proximal region.

7. The femoral stem device of claim 4, wherein the partial hollowing comprises beams laterally extending from the central spine.

8. The femoral stem device of claim 7, wherein the central spine and beams define a series of intervening hollowed spaces.

9. The femoral stem device of claim 8, wherein the intervening hollowed spaces comprise laterally opposed internal surfaces.

10. The femoral stem device of claim 8, wherein the intervening hollowed spaces comprise longitudinally opposed internal surfaces.

11. The femoral stem device of claim 1, wherein the deformation transfers at least a portion of the loading into a femur.

12. The femoral stem device of claim 1, wherein the walls comprise a width of 2 mm.

13. The femoral stem device of claim 1, wherein the stem is configured for implantation into a femur.

14. The femoral stem device of claim 1, wherein the distal region of the stem is solid.

15. The femoral stem device of claim 14, wherein the solid stem is partially hollowed to provide a channel for removal of unfused alloy powder.

16. The femoral stem device of claim 1, wherein the neck is configured for attachment to a femoral head.

17. The femoral stem device of claim 1, wherein the femoral stem device comprises Titanium grade 5 (Ti-6Al-4V).
